(19) United States
(12) Patent Application Publication
(10) Pub. No.: US 2022/0275079 A1
(43) Pub. Date: Sep. 1, 2022

(54) Clonal Hematopoiesis and Risk of Chronic Liver Disease

(71) Applicants: The General Hospital Corporation, Boston, MA (US); Dana-Farber Cancer Institute, Inc., Boston, MA (US)

(72) Inventors: Connor Emdin, Boston, MA (US); Waihay Wong, Boston, MA (US); Benjamin L. Ebert, Boston, MA (US); Pradeep Natarajan, Boston, MA (US)

(21) Appl. No.: 17/529,045

(22) Filed: Nov. 17, 2021

Related U.S. Application Data
(60) Provisional application No. 63/213,613, filed on Jun. 22, 2021.
(60) Provisional application No. 63/116,382, filed on Nov. 20, 2020.

(51) Int. Cl. C07K 16/24; A61K 31/4045; A61K 31/573; A61K 31/737; A61K 38/20; A61K 31/4748; A61K 31/416; A61P 1/16; C12Q 1/6883

(52) U.S. Cl. CPC C07K 16/248; A61K 31/4045; A61K 31/573; A61K 31/737; A61K 38/2006; C07K 16/245; A61K 31/4748; A61K 31/416; A61P 1/16; C12Q 1/6883; C12Q 2600/156

(57) ABSTRACT

This application provides for methods of treatment for liver disease, especially in subjects with a DNMT3A, TET2, JAK2, and/or ASXL1. The application also provides for methods of diagnosing liver disease, as well as kits for use in the claimed methods.

### CROSS-REFERENCE TO RELATED APPLICATION

This application claims the benefit and priority of U.S. Provisional Application No. 63/116,382, filed Nov. 20, 2020, and U.S. Provisional Application No. 63/213,613, filed Jun. 22, 2021, which are incorporated by reference herein in their entireties for any purpose.

### FIELD

Methods of preventing, treating, and diagnosing clonal hematopoiesis and chronic liver disease.

### BACKGROUND

Chronic liver disease affects up to 19% of Americans in an age-dependent fashion. This disease encompasses a spectrum of histopathologic changes that progress from accumulation of liver fat (steatosis) to liver inflammation with hepatocyte injury (steatohepatitis), fibrosis and cirrhosis. Chronic liver disease is characterized by an inflammatory and fibrotic response to an initial insult, most commonly steatosis from excess alcohol consumption or from obesity or liver injury from viral infection. Scaglione et al., The epidemiology of cirrhosis in the United States: A population-based study, J. Clin. Gastroenterol., 49:690-696 (2015). However, the factors promoting progression from steatosis to inflammation and fibrosis are poorly understood.

Liver inflammation and fibrosis are mediated in part by nonparenchymal cells of the liver, including sinusoidal endothelial cells, dendritic cells, lymphocytes, and macrophages. Resident liver macrophages (Kupffer cells) and bone marrow derived monocytes and macrophages have been implicated in responses to liver injury in both murine models and humans. Brempelis et al., Infiltrating monocytes in liver injury and repair, Clin. Transl. Immunology 5:e113 (2016); Krenkel et al., Liver macrophages in tissue homeostasis and disease, Nat. Rev. Immunol. 17:306-321 (2017). In non-alcoholic fatty liver disease (NAFLD), macrophage recruitment is required for progression to non-alcoholic steatohepatitis (NASH), whereas inhibition of monocyte recruitment prevents progression in mouse models. Krenkel et al., Therapeutic inhibition of inflammatory monocyte recruitment reduces steatohepatitis and liver fibrosis, Hepatology 67:1270-1283 (2018); Krenkel et al., Myeloid cells in liver and bone marrow acquire a functionally distinct inflammatory phenotype during obesity-related steatohepatitis, Gut 69(3):551-563 (2019); Baeck et al., Pharmacological inhibition of the chemokine CCL2 (MCP-1) diminishes liver macrophage infiltration and steatohepatitis in chronic hepatic injury, Gut 61:416-426 (2012). Furthermore, monocyte-derived inflammatory macrophages are enriched in liver samples from patients who progress from NASH to cirrhosis. Krenkel et al., Therapeutic inhibition of inflammatory monocyte recruitment reduces steatohepatitis and liver fibrosis, Hepatology 67:1270-1283 (2018).

Dysregulated inflammatory responses can occur in the setting of clonal hematopoiesis of indeterminate potential (CHIP), which is characterized by the expansion of hematopoietic cells bearing oncogenic somatic mutations, most frequently in the genes DNMT3A, TET2, and ASXL1. Steensma et al., Clonal hematopoiesis of indeterminate potential and its distinction from myelodysplastic syndromes, Blood 126(1):9-16 (2015), doi:10.1182/blood-2015-03-631747. Whole exome sequence (WES) analysis of blood DNA has led to the recognition that CHIP is a common phenomenon with increasing prevalence in older age, present in greater than 10% of persons over the age of 70. Genovese et al., Clonal hematopoiesis and blood-cancer risk inferred from blood DNA sequence, N. Engl. J. Med. 371:2477-2487 (2014); Jaiswal et al., Age-related clonal hematopoiesis associated with adverse outcomes, N. Engl. J. Med. 371L2488-2498 (2014); Xie et al., Age-related mutations associated with clonal hematopoietic expansion and malignancies, Nat. Med. 20:1472-1478 (2014). CHIP is associated with future risk of hematologic malignancy, all-cause mortality, and atherosclerotic cardiovascular disease. Genovese et al., Clonal hematopoiesis and blood-cancer risk inferred from blood DNA sequence, N. Engl. J. Med. 371:2477-2487 (2014); Jaiswal et al., Age-related clonal hematopoiesis associated with adverse outcomes, N. Engl. J. Med. 371L2488-2498 (2014); Jaiswal et al., Clonal hematopoiesis and risk of atherosclerotic cardiovascular disease, N. Engl. J. Med. 377:111-121 (2017); Bick et al., Genetic interleukin 6 signaling deficiency attenuates cardiovascular risk in clonal hematopoiesis, Circulation 141:124-131 (2020). Murine models have revealed the pro-inflammatory role of macrophages derived from mutant CHIP clones and their contributions to atherogenesis. Jaiswal et al., Clonal hematopoiesis and risk of atherosclerotic cardiovascular disease, N. Engl. J. Med. 377:111-121 (2017); Fuster et al., Clonal hematopoiesis associated with TET2 deficiency accelerates atherosclerosis development in mice, Science 335, 842-847 (2017); Sano et al., Tet2-mediated clonal hematopoiesis accelerates heart failure through a mechanism involving the IL-1β/NLRP3 inflammasome, J. Am. Coll. Cardiol. 71:875-886 (2018). Given the pervasive nature of circulating immune cells, CHIP could potentially influence the trajectory of steatohepatitis and cirrhosis through aberrant inflammation in the liver.

### SUMMARY

The present disclosure provides methods that relate to preventing, treating and diagnosing liver disease in a human subject. In some embodiments, the method of treating liver disease comprises treating the liver disease with lifestyle modifications and/or by administering an effective amount of at least one pharmaceutical agent for treating liver disease, wherein the subject has a DNMT3A, TET2, JAK2, and/or ASXL1 mutation, thereby preventing and treating liver disease that relate to the presence of one or more mutations in one or more genes associated with CHIP. A list of CHIP-associated genes that may be evaluated for mutations is found in Tables 1 and 3.

In some embodiments, the method of treating liver disease in a human subject comprises: (a) sequencing at least part of a genome comprising DNMT3A, TET2, JAK2, and/or ASXL1 of one or more cells in a blood sample of the subject; (b) determining from the sequencing whether the subject has one or more mutations in DNMT3A, TET2, JAK2, and/or ASXL1, and (c) if it is determined that the subject has at least one DNMT3A, TET2, JAK2, and/or ASXL1 mutation, treating the liver disease with lifestyle modifications and/or by administering an effective amount of at least one pharmaceutical agent for treating liver disease, to the subject thereby treating the liver disease.

In some embodiments, the pharmaceutical agent for treating liver disease targets NLRP3 inflammasome, IL-1β, IL-6, IL-6 receptor, CCL22, MCP1, and/or MIP2. In some embodiments, the pharmaceutical agent is an inhibitor of NLRP3 inflammasome, IL-1β, IL-6, IL-6 receptor, CCL22, MCP1, and/or MIP2. In some embodiments, the pharmaceutical agent is an antibody or an antigen binding fragment thereof. In some embodiments, the pharmaceutical agent may comprise melatonin, methylprednisolone, AC-201, clazakizumab, tocilizumab, anakinra (Kineret®), canakinumab (Ilaris®), ziltivekimab, sarilumab, sinomenine, fucoidan, and bindarit.

In some embodiments, the pharmaceutical agent comprises a hepatitis treatment. In some embodiments, the hepatitis treatment may comprise sofosbuvir, ribavirin, pegylated interferon, interferon alpha, daclatasvir, adefovir, entecavir, lamivudine, telbivudine, tenofovir, elbasvir-grazoprevir, ledipasvir-sofosbuvir, sofosbuvir-velpatasvir, ombitasvir-paritaprevir-ritonavir, ombitasvir-paritaprevir-ritonavir-dasabuvir, and/or a vaccine.

In some embodiments, the pharmaceutical agent comprises a treatment to prevent ascites, edema, portal hypertension, severe bleeding, or infections.

In some embodiments, the method of treating liver disease comprises at least one lifestyle modification. In some embodiments, at least one lifestyle modification is chosen from weight loss, exercise, and dietary modification. In some embodiments, dietary modification is chosen from cessation or reduction of alcohol consumption, a low sodium diet, a low fat diet, a low carbohydrate diet, a high fiber diet, and cessation or reduction of medications and/or supplements that are toxic to the liver.

In some embodiments, the method for diagnosing liver disease, diagnosing CHIP, predicting risk for CHIP, and/or targeted prevention of liver disease in a human subject comprises: (a) obtaining a nucleic acid sample from the subject; (b) detecting whether the sample contains at least one DNMT3A, TET2, JAK2, and/or ASXL1 mutation; and (c) diagnosing the subject as having liver disease, CHIP, a risk for CHIP, and/or a risk of liver disease when at least one DNMT3A, TET2, JAK2, and/or ASXL1 mutation is detected. In some embodiments, the DNMT3A, TET2, JAK2, and/or ASXL1 mutation is a loss-of-function mutation.

In some embodiments, the DNMT3A, TET2, JAK2, and/or ASXL1 mutation comprises an amino acid change in TET2 chosen from S145N, S282F, A308T, N312S, L346P, P399L, S460F, D666G, S817T, P941S, C1135Y, R1167T, I1175V, S1204C, R1214W, D1242R, D1242V, Y1245S, R1261C, R1261H, R1261L, F1287L, W1291R, K1299E, K1299N, R1302G, E1318G, P1367S, C1396W, L1398R, V1417F, G1869W, L1872P, I1873T, C1875R, H1881Q, H1881R, R1896M, R1896S, S1898F, V1900A, G1913D, A1919V, R1926H, P1941S, P1962L, R1966H, R1974M, and R2000K.

In some embodiments, the DNMT3A, TET2, JAK2, and/or ASXL1 mutation comprises an amino acid change in JAK2 of V617F.

In some embodiments, a TP53, SF3B1, SRSF2, GNB1, CBL and/or PPM1D mutation is also detected in the human subject.

In some embodiments, at least one mutation is detected by a SNP array.

In some embodiments, the human subject does not have a mutation in NLRP3, IL-1β, and/or IL-6 that inhibits the NLRP3/IL-1β/IL-6 signaling blockade. In some embodiments, the human subject does not have a D358A mutation in IL6R.

In some embodiments, the subject also exhibits one or more risk factors comprising increased levels of cholesterol, increased levels of liver iron, increased alcohol consumption, increased body mass index, and/or obesity.

In some embodiments, the human subject does not have myeloproliferative neoplasm. In some embodiments, the human subject does not have portal vein thrombosis.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C provide data showing clonal hematopoiesis of indeterminate potential (CHIP) is associated with chronic liver disease. FIG. 1A shows association of CHIP with prevalent chronic liver disease. FIG. 1B shows association of CHIP with subtypes of incident chronic liver disease in UK Biobank. FIG. 1C shows association of clonal hematopoiesis with NASH in MGB Biobank. Estimates in prevalent analyses were derived using logistic regression, with adjustment for age and sex. Estimates in incident analyses were derived using Cox proportional hazards regression, with adjustment for age and sex. NASH was defined as chronic liver disease amount individuals with a body mass index of 30 kg/m² or more who consumed more than 21 drinks per week for men and more than 14 drinks per week for women. Alcohol-related liver disease was defined as chronic liver disease among individuals who consumed 21 drinks or less per week for men and 14 drinks or less per week for women. ARIC, Atherosclerosis Risk in Communities; CI, 95% confidence interval; FHS, Framingham Heart Study; OR, odds ratio; UKBB, UK Biobank; MGB, Massachusetts General Brigham.

FIGS. 2A-B show cumulative risk of chronic liver disease by clonal hematopoiesis status in UK Biobank. Cumulative risk of chronic liver disease by age was modeled using Cox proportional hazards model with age as the underlying time variable and adjustment for sex. CHIP, clonal hematopoiesis of indeterminate potential. FIG. 2A shows cumulative risk of chronic liver disease by age. In FIG. 2B, patients with clonal hematopoiesis (CHIP) had a cumulative risk of 5.0 while patients without (no CHIP) had a cumulative risk of 1.1%; p<0.001.

FIGS. 3A-D show the prevalence of liver inflammation and hepatic steatosis (fatty liver) based on magnetic resonance imaging of individuals in the UK Biobank. FIGS. 3A and 3B show the prevalence of liver inflammation and hepatic steatosis, respectively, on magnetic resonance imaging among 8251 individuals in the UK Biobank. FIG. 3C shows a Perspectum LiverMultiScan™ corrected T1 image of a patient with CHIP. FIG. 3D shows a Perspectum LiverMultiScan™ proton density fat fraction of a patient with CHIP. Images were reproduced with permission of UK Biobank. Liver inflammation was defined as a corrected T1 signal≥795 ms. Fatty liver was defined as a proton density fat fraction≥5%. Logistic regression, with adjustment for age and sex, was used to test the association between CHIP status and the presence of fatty liver and liver inflammation. CHIP, clonal hematopoiesis of indeterminate potential; cT1, corrected T1; PDFF, proton density fat fraction.

FIG. 4 shows Mendelian randomization estimates of the association of CHIP with chronic liver disease. Estimates were derived using MR-RAPS (Mendelian Randomization using Robust Adjusted Profile Score) with 184 independent genetic variants with significance of p<0.0001.

FIGS. 5A-I show increased steatohepatitis and liver fibrosis in Tet2^{-/-} transplanted mice after a Western diet. FIGS. 5A-D: Ldlr^{-/-} mice were transplanted with Tet2^{-/-} (n=25) or control (n=20) bone marrow, and fed a Western diet for 10 weeks. They were graded histologically for steatosis (FIG. 5A), inflammation (FIG. 5B), hepatocyte ballooning injury (FIG. 5C), and compiled into a NASH activity score (NAS) (FIG. 5D). FIGS. 5E-H show photomicrographs of graded histologic features including steatosis (FIG. 5E), inflammation (FIG. 5F), hepatocyte ballooning injury (FIG. 5G, arrowhead), and apoptosis (FIG. 5H, arrowhead). In FIG. 5I, Masson's trichrome staining demonstrates absence of perivenular fibrosis in control and Tet2^{-/-} transplanted mice.

FIGS. 6A-R show increased steatohepatitis, liver fibrosis, and proinflammatory cytokine levels in Tet2^{-/-} transplanted mice. FIGS. 6A-D: B6.SJL mice were transplanted with Tet2^{-/-} (n=20), Tet2^{-/}Nlrp3^{-/-} (n=10), or control (n=13) bone marrow cells, and fed CDAHFD for 11 weeks. Compared to control and Tet2^{-/-} Nlrp3^{-/-} transplanted animals, Tet2^{-/-} transplanted mice show similar accumulation of liver fat (FIG. 6A), and show increased lymphocyte and macrophage inflammation (FIG. 6B) and hepatocyte ballooning injury (FIG. 6C). Steatohepatitis was graded using modified NASH CRN histologic criteria. Increased overall NASH activity was observed in Tet2^{-/-} transplanted mice (FIG. 6D). FIGS. 6E-H show photomicrographs of graded histologic features including steatosis (FIG. 6E), inflammation (FIG. 6F-G), and hepatocyte ballooning injury (FIG. 6H). FIGS. 6I-J: B6.SJL mice were transplanted with Tet2^{-/-} (n=30) or control (n=13) bone marrow cells, and fed CDAHFD for 19 weeks. Compared to control animals, Tet2^{-/-} transplanted mice show increased collagen fibrosis by picosirius red staining (FIG. 6I) with a statistically significant difference on image quantification (FIG. 6J). FIGS. 6K-N: after 19 weeks of CDAHFD, Tet2^{-/-} or control transplanted mice were bled and serum obtained for cytokine measurements. FIGS. 6O-R: bone marrow derived macrophages from Tet2^{-/-}, Tet2^{-/}Nlrp^{3-/-} or control mice were primed with low dose lipopolysaccharide for 2 hours and stimulated with palmitic acid or cholesterol monohydrate crystals as indicated for 6 hours.

FIGS. 7A-C show gene expression analysis of sorted liver macrophages and bulk liver RNA in Tet2^{-/-} (Tet2KO) transplanted mice fed CDAHFD. FIG. 7A shows unsupervised hierarchical clustering of differentially regulated genes in sorted liver macrophages from B6.SJL mice transplanted with Tet2^{-/-} (n=4) versus control (n=4) bone marrow cells and fed CDAHFD for 11 weeks. Gene set enrichment analysis in FIG. 7B shows enrichment of hallmark pathways (upper panel) and macrophage-related immunologic signatures (lower panel) in sorted liver macrophages from Tet2^{-/-} transplanted mice fed CDAHFD for 11 weeks. The total number of enriched genes and most highly enriched genes in each signature are shown. FIG. 7C shows selected enriched gene sets in bulk livers of Tet2^{-/-} transplanted mice fed CDAHFD. The most highly enriched genes in each signature are shown. NES, normalized enrichment score.

FIGS. 8A-N show increased or persistent steatohepatitis in Dnmt3a^{-/-} or Tet2^{-/-} transplanted mice, respectively. FIGS. 8A-E: B6.SJL mice were transplanted with Dnmt3a^{-/-} (n=24) or control (n=19) bone marrow cells, and fed CDAHFD for 11 weeks. Steatohepatitis was graded using NASH CRN histologic scoring criteria for steatosis (FIG. 8A), inflammation (FIG. 8B), and hepatocyte ballooning injury (FIG. 8C), and integrated into a composite NASH activity score (FIG. 8D). Collagen fibrosis was measured by Masson's trichrome staining (FIG. 8E). FIGS. 8F-J: B6.SJL mice were transplanted with Tet2^{-/-} (n=21) or control (n=15) bone marrow cells, and fed CDAHFD for 11 weeks, after which mice were switched to regular diet for 10 days. Compared to control animals, Tet2^{-/-} transplanted mice show similar resolution of liver fat (FIG. 8F) but show persistent inflammation (FIG. 8G) and occasional hepatocyte ballooning injury (FIG. 8H), such that residual NASH activity score was higher (FIG. 8I). Collagen fibrosis, as measured by Masson's trichrome staining, was not significantly different (FIG. 8J). FIGS. 8K-N: Immunohistochemical stains demonstrate the presence of F4/80+CD45.2₊CD45.1₋ macrophages in livers of bone marrow transplanted mice fed CDAHFD.

### DESCRIPTION OF THE EMBODIMENTS

### I. Methods of Treatment

The present application includes methods of prevention and treatment of liver disease as it relates to one or more loss-of-function mutations in the genes DNMT3A, TET2, JAK2, and/or ASXL1. These mutations are found in CHIP and are associated with increased risk for chronic liver diseases, such as liver fibrosis and cirrhosis.

In some embodiments, a method of preventing and treating liver disease in a human subject comprises (a) sequencing at least part of a genome comprising DNMT3A, TET2, JAK2, and/or ASXL1 of one or more cells in a blood sample of the subject; (b) determining from the sequencing whether the subject has one or more mutations in DNMT3A, TET2, JAK2, and/or ASXL1; and (c) if it is determined that the subject has at least one DNMT3A, TET2, JAK2, and/or ASXL1 mutation, treating the liver disease with lifestyle modifications and/or by administering an effective amount of at least one pharmaceutical agent for treating liver disease, to the subject thereby treating the liver disease.

A. DNMT3A Mutations

DNMT3A mutations, either alone or in combination with other indicators, may cause or be associated with liver disease. The DNMT3A gene encodes for an enzyme called DNA methyltransferase 3 alpha which is involved in DNA methylation. The enzyme establishes methylation patterns in hematopoietic stem cells, promoting their differentiation into blood cell types. As DNMT3A is an enzyme that alters DNA methylation, it is believed that perturbing its function results in an abnormal epigenetic state.

One or more than one DNMT3A mutation may be present in a somatic blood cell clone. A DNMT3A mutation may be a frameshift mutation, a nonsense mutation, a missense mutation, or a splice-site variant mutation. A DNMT3A mutation may also be a loss-of-function DNMT3A mutation.

In some embodiments, a mutation in DNMT3A leads to non-expression or decreased expression of the DNMT3A protein or expression of a truncated or non-functional form of the DNMT3A protein. In some embodiments, a mutation in DNMT3A leads to a change in the structure or function of the DNMT3A protein.

In some embodiments, the mutation in DNMT3A is a frameshift mutation. In some embodiments, the frameshift mutation is caused by insertion or deletion of a number of nucleotides that is not divisible by three. The mutation in DNMT3A may also be an insertion or deletion of a number of nucleotides that is divisible by three, wherein one or more amino acids are added or deleted from the wild-type DNMT3A amino acid sequence.

In some embodiments, the mutation in DNMT3A is a nonsense mutation. In some embodiments, the nonsense mutation is a point mutation (i.e., single nucleotide change) that results in a premature stop codon or a nonsense codon (i.e., a codon that does not code for an amino acid) in the transcribed RNA. In some embodiments, the nonsense mutation leads to a truncated, incomplete and/or nonfunctional DNMT3A protein.

In some embodiments, the mutation in DNMT3A is a missense mutation. In some embodiments, the missense mutation is a point mutation that codes for a different amino acid than that found in the wildtype DNMT3A sequence. In some embodiments, the missense mutation is within nucleotides that encode one of the catalytic domains of the DNMT3A protein.

B. TET2 Mutations

TET2 mutations, either alone or in combination with other indicators, may cause or be associated with liver disease. As Tet2 is an enzyme that alters DNA methylation, it is believed that perturbing its function results in an abnormal epigenetic state. For example, TET2 converts 5-methylcytosine to 5-hydroxymethylcytosine, which ultimately leads to demethylation. As methylation at promoters and enhancers anti-correlates with gene expression and transcription factor binding, TET2 mutations can impact gene expression and transcription factor binding.

One or more than one TET2 mutation may be present in a somatic blood cell clone. A TET2 mutation may be a frameshift mutation, a nonsense mutation, a missense mutation, or a splice-site variant mutation. A TET2 mutation may also be a loss-of-function TET2 mutation.

In some embodiments, a mutation in TET2 leads to non-expression or decreased expression of the TET2 protein or expression of a truncated or non-functional form of the TET2 protein. In some embodiments, a mutation in TET2 leads to a change in the structure or function of the TET2 protein.

In some embodiments, the mutation in TET2 is a frameshift mutation. In some embodiments, the frameshift mutation is caused by insertion or deletion of a number of nucleotides that is not divisible by three. The mutation in TET2 may also be an insertion or deletion of a number of nucleotides that is divisible by three, wherein one or more amino acids are added or deleted from the wild-type TET2 amino acid sequence.

In some embodiments, the mutation in TET2 is a nonsense mutation. In some embodiments, the nonsense mutation is a point mutation (i.e., single nucleotide change) that results in a premature stop codon or a nonsense codon (i.e., a codon that does not code for an amino acid) in the transcribed RNA. In some embodiments, the nonsense mutation leads to a truncated, incomplete and/or nonfunctional TET2 protein.

In some embodiments, the mutation in TET2 is a missense mutation. In some embodiments, the missense mutation is a point mutation that codes for a different amino acid than that found in the wildtype TET2 sequence. In some embodiments, the missense mutation is within nucleotides that encode one of the catalytic domains of the TET2 protein. In some embodiments, the missense mutation causes a change in amino acid from that encoded by the wildtype sequence at amino acids 1104-1481 or 1843-2002 of the TET2 protein.

In some embodiments, the mutation in TET2 results in an amino acid change in TET2 chosen from at least one of S145N, S282F, A308T, N312S, L346P, P399L, S460F, D666G, S817T, P941S, C1135Y, R1167T, I1175V, S1204C, R1214W, D1242R, D1242V, Y1245S, R1261C, R1261H, R1261L, F1287L, W1291R, K1299E, K1299N, R1302G, E1318G, P1367S, C1396W, L1398R, V1417F, G1869W, L1872P, I1873T, C1875R, H1881Q, H1881R, R1896M, R1896S, S1898F, V1900A, G1913D, A1919V, R1926H, P1941S, P1962L, R1966H, R1974M, and R2000K.

C. JAK2 Mutations

JAK2 mutations, either alone or in combination with other indicators, may cause or be associated with liver disease. JAK2 encodes for a non-receptor tyrosine kinase involved in various processes such as cell growth, development, differentiation or histone modifications. The JAK2 kinase plays a role in innate and adaptive immunity; in signal transduction via association with (1) type I receptors such as growth hormone, prolactin, leptin, erythropoietin, and thrombopoietin, or (2) type II receptors such as IFN-α, IFN-β, IFN-γ, and interleukins. JAK2 is especially important for controlling the production of blood cells from hematopoietic stem cells. As a key player in myriad signaling pathways, it is likely that perturbing JAK2's function results in aberrant downstream signaling cascades.

One or more than one JAK2 mutation may be present in a somatic blood cell clone. A JAK2 mutation may be a frameshift mutation, a nonsense mutation, a missense mutation, or a splice-site variant mutation. A JAK2 mutation may also be a loss-of-function JAK2 mutation.

JAK2 gain-of-function mutations are known to cause myeloproliferative neoplasms (MPNs; previously termed myeloproliferative disorders, MPDs) that can lead to portal vein thrombosis (PVT), mesenteric vein thrombosis (MVT), or splanchnic vein thrombosis (SVT). Colaizza et al., The JAK2 V617F mutation frequently occurs in patients with portal and mesenteric venous thrombosis. J. Thromb. Haemost. 5:55-61 (2007); DeLeve et al., Vascular disorders of the liver, Hepatology 49(5):1729-1764 (2009); Martinelli et al., Rare thromboses of cerebral, splanchnic and upper-extremity veins: a narrative review, Thromb. Haemost. 103(6):1136-1144 (2010); Smallberg et al., The JAK2 46/1 haplotype in Budd-Chiari syndrome and portal vein thrombosis, Blood 117(5):3968-73 (2011). The V617F gain-of-function mutation is found in most MPD patients; it is an acquired somatic event in sporadic MPDs that leads to a constitutive activation of the JAK-signal transducer and activator of transcription signal transduction pathway. Baxter et al., Acquired mutation of the tyrosine kinase JAK2 in human myeloproliferative disorders, Lancet 365:1054-61 (2005); James et al., A unique clonal JAK 2 mutation leading to constitutive signaling causes polycythaemia vera, Nature 434:1144-8 (2005); Kralovics et al., Altered gene expression in myeloproliferative disorders correlates with activation of signaling by the V617F mutation of Jak2, Blood 106:3374-6. Levine et al., Activating mutation in the tyrosine kinase JAK2 in polycythemia vera, essential thrombocythemia, and myeloid metaplasia with myelofibrosis, Cancer Cell 7L387-97 (2005); Martinelli et al., Rare thromboses of cerebral, splanchnic and upper-extremity veins: a narrative review, Thromb. Haemost. 103(6):1136-1144 (2010). MPD-related JAK2 mutations are distinct from JAK2 loss-of-function mutations in CHIP and CHIP-associated liver disease.

In many embodiments of the present application, the JAK2 mutation is a loss-of-function mutation. In many embodiments, the JAK2 mutation is not a gain-of-function mutation. In many embodiments, a human subject with a JAK2 mutation does not have myeloproliferative neoplasm. In many embodiments, a human subject with a JAK2 mutation does not have portal vein thrombosis.

In some embodiments, a mutation in JAK2 leads to non-expression or decreased expression of the JAK2 protein or expression of a truncated or non-functional form of the JAK2 protein. In some embodiments, a mutation in JAK2 leads to a change in the structure or function of the JAK2 protein.

In some embodiments, the mutation in JAK2 is a frameshift mutation. In some embodiments, the frameshift mutation is caused by insertion or deletion of a number of nucleotides that is not divisible by three. The mutation in JAK2 may also be an insertion or deletion of a number of nucleotides that is divisible by three, wherein one or more amino acids are added or deleted from the wild-type JAK2 amino acid sequence.

In some embodiments, the mutation in JAK2 is a nonsense mutation. In some embodiments, the nonsense mutation is a point mutation (i.e., single nucleotide change) that results in a premature stop codon or a nonsense codon (i.e., a codon that does not code for an amino acid) in the transcribed RNA. In some embodiments, the nonsense mutation leads to a truncated, incomplete and/or nonfunctional JAK2 protein.

In some embodiments, the mutation in JAK2 is a missense mutation. In some embodiments, the missense mutation is a point mutation that codes for a different amino acid than that found in the wildtype JAK2 sequence. In some embodiments, the missense mutation is within nucleotides that encode one of the catalytic domains of the JAK2 protein.

In some embodiments, the mutation in JAK2 results in an amino acid change in JAK2 of V617F.

D. ASXL1 Mutations

ASXL1 mutations, either alone or in combination with other indicators, may cause or be associated with liver disease. The ASXL1 protein is an epigenetic regulator that binds to chromatin and influences gene expression via DNA methylation and ubiquitination. Thus, it is likely that perturbing ASXL1's function results in an abnormal epigenetic state.

One or more than one ASXL1 mutation may be present in a somatic blood cell clone. An ASXL1 mutation may be a frameshift mutation, a nonsense mutation, a missense mutation, or a splice-site variant mutation. An ASXL1 mutation may also be a loss-of-function ASXL1 mutation.

In some embodiments, a mutation in ASXL1 leads to non-expression or decreased expression of the ASXL1 protein or expression of a truncated or non-functional form of the ASXL1 protein. In some embodiments, a mutation in ASXL1 leads to a change in the structure or function of the ASXL1 protein.

In some embodiments, the mutation in ASXL1 is a frameshift mutation. In some embodiments, the frameshift mutation is caused by insertion or deletion of a number of nucleotides that is not divisible by three. The mutation in ASXL1 may also be an insertion or deletion of a number of nucleotides that is divisible by three, wherein one or more amino acids are added or deleted from the wild-type ASXL1 amino acid sequence.

In some embodiments, the mutation in ASXL1 is a nonsense mutation. In some embodiments, the nonsense mutation is a point mutation (i.e., single nucleotide change) that results in a premature stop codon or a nonsense codon (i.e., a codon that does not code for an amino acid) in the transcribed RNA. In some embodiments, the nonsense mutation leads to a truncated, incomplete and/or nonfunctional ASXL1 protein.

In some embodiments, the mutation in ASXL1 is a missense mutation. In some embodiments, the missense mutation is a point mutation that codes for a different amino acid than that found in the wildtype ASXL1 sequence. In some embodiments, the missense mutation is within nucleotides that encode one of the catalytic domains of the ASXL1 protein.

E. Other Mutations

In some embodiments, other mutations may be detected in addition to at least one mutation in DNMT3A, TET2, JAK2, and/or ASXL1. In some embodiments, the mutation is chosen from at least one mutation in Th53, SF3B1, SRSF2, GATB1, CBL, and/or PPM1D. In some embodiments, the mutation is chosen from at least one mutation shown in Table 1 below.

TABLE 1
CHIP variants ascertained through whole-exome sequencing.

| Gene name | Mutation(s) | Accession No. |
|---|---|---|
| ASXL1 | Frameshift/nonsense/splice-site in exon 11-12 | NM_015338 |
| ASXL2 | Frameshift/nonsense/splice-site in exon 11-12 | NM_018263 |
| BCOR | Frameshift/nonsense/splice-site | NM_001123385 |
| BCORL1 | Frameshift/nonsense/splice-site | NM_021946 |
| BRAF | G464E, G464V, G466E, G466V, G469R, G469E, | NM_004333 |
| | G469A, G469V, V471F, V472S, L485W, N581S, I582M, | |
| | I592M, I592V, D594N, D594G, D594V, D594E, | |
| | F595L, F595S, G596R, L597V, L597S, L597Q, L597R, | |
| | A598V, V600M, V600L, V600K, V600R, V600E, | |
| | V600A, V600G, V600D, K601E, K601N, R603*, | |
| | W604R, W604G, S605G, S605F, S605N, G606E, | |
| | G606A, G606V, H608R, H608L, G615R, S616P, S616F, | |
| | L618S, L618W | |
| BRCC3 | Frameshift/nonsense/splice-site | NM_024332 |
| CBL | RING finger missense p.381-421 | NM_005188 |
| CBLB | RING finger missense p.372-412 | NM_170662 |
| CEBPA | Frameshift/nonsense/splice-site | NM_004364 |
| CREBBP | Frameshift/nonsense/splice-site, D1435E, R1446L, | NM_004380 |
| | R1446H, R1446C, Y1450C, P1476R, Y1482H, H1487Y, | |
| | W1502C, Y1503D, Y1503H, Y1503F, S1680del | |
| CSF1R | L301F, L301S, Y969C, Y969N, Y969F, Y969H, Y969D | NM_005211 |
| CSF3R | T615A, T618I, truncating c.741-791 | NM_000760 |
| CTCF | Frameshift/nonsense, R377C, R377H, P378A, P378L | NM_006565 |
| CUX1 | Frameshift/nonsense | NM_181552 |
| DNMT3A | Frameshift/nonsense/splice-site, F290I, F290C, V296M, | NM_022552 |
| | P307S, P307R, R326H, R326L, R326C, R326S, G332R, | |
| | G332E, V339A, V339M, V339G, L344Q, L344P, | |
| | R366P, R366H, R366G, A368T, A368V, R379H, R379C, | |
| | I407T, I407N, I407S, F414L, F414S, F414C, A462V, | |
| | K468R, C497G, C497Y, Q527H, Q527P, Y533C, S535F, | |
| | C537G, C537R, G543A, G543S, G543C, L547H, L547P, | |
| | L547F, M548I, M548K, G550R, W581R, W581G, | |
| | W581C, R604Q, R604W, R635W, R635Q, S638F, | |
| | G646V, G646E, L653W, L653F, I655N, V657A, | |
| | V657M, R659H, Y660C, V665G, V665L, M674V, | |
| | R676W, R676Q, G685R, G685E, G685A, D686Y, | |
| | D686G, R688H, G699R, G699S, G699D, P700L, P700S, | |
| | P700R, P700Q, P700T, P700A, D702N, D702Y, | |
| | V704M, V704G, I705F, I705T, I705S, I705N, G707D, | |
| | G707V, C710S, C710Y, S714C, V716D, V716F, V716I, | |
| | N717S, N717I, P718L, R720H, R720G, K721R, K721T, | |
| | Y724C, R729Q, R729W, R729G, F731C, F731L, F731Y, | |
| | F731I, F732del, F732C, F732S, F732L, E733G, E733A, | |
| | F734L, F734C, Y735C, Y735N, Y735S, R736H, R736C, | |
| | R736P, L737H, L737V, L737F, L737R, A741V, P742P, | |
| | P743R, P743L, R749C, R749L, R749H, R749G, F751L, | |
| | F751C, F752del, F752C, F752L, F752I, F752V, W753G, | |
| | W753C, W753R, L754P, L754R, L754H, F755S, F755I, | |
| | F755L, M761I, M761V, G762C, V763I, S770L, S770W, | |
| | S770P, R771Q, F772I, F772V, L773R, L773V, E774K, | |
| | E774D, E774G, I780T, D781G, R792H, W795C, | |
| | W795L, G796D, G796V, N797Y, N797H, N797S, | |
| | P799S, P799R, P799H, R803S, R803W, P804L, P804S, | |
| | K826R, S828N, K829R, T835M, N838D, K841Q, | |
| | Q842E, P849L, D857N, W860R, E863D, F868S, | |
| | G869S, G869V, M880V, S881R, S881I, R882H, R882P, | |
| | R882C, R882G, A884P, A884V, Q886R, L889P, L889R, | |
| | G890D, G890R, G890S, V895M, P896L, V897G, | |
| | V897D, R899L, R899H, R899C, L901R, L901H, P904L, | |
| | F909C, P904Q, A910P, C911R, C911Y | |
| EED | Frameshift/nonsense/splice-site, L240Q, I363M | NM_003797 |
| EP300 | Frameshift/nonsense/splice_site, VF1148_1149del, | NM_001429 |
| | D1399N, D1399Y, P1452L, Y1467N, Y1467H, Y1467C, | |
| | R1627W, A1629V | |
| ETNK1 | N244S, N244T, N244K | NM_018638 |
| ETV6 | Frameshift/ nonsense/splice-site | NM_001987 |
| EZH2 | Frameshift/nonsense/splice-site, Q62R, N102S, F145S, | NM_001203247 |
| | F145C, F145Y, F145L, G159R, E164D, R202Q, K238E, | |
| | E244K, R283Q, H292R, P488S, R497Q, R561H, T568I, | |
| | K629E, Y641N, Y641H, Y641S, Y641C, Y641F, | |
| | D659Y, D659G, V674M, A677G, A677V, R679C, | |
| | R679H, R685C, R685H, A687V, N688I, N688K, | |
| | H689Y, S690P, I708V, I708T, I708M, E720K, E740K | |
| FLT3 | V579A, V592A, V592I, F594L, FY590-591GD, D835Y, | NM_004119 |
| | D835H, D835E, del835 | |
| GATA1 | Frameshift/nonsense/splice-site | NM_002049 |
| GATA2 | Frameshift/nonsense/splice-site, R293Q, N317H, | NM_001145661 |
| | A318T, A318V, A318G, G320D, L321P, L321F, L321V, | |
| | Q328P, R330Q, R361L, L359V, A372T, R384G, R384K | |
| GATA3 | Frameshift/nonsense/splice-site ZNF domain, R276W, | NM_001002295 |
| | R276Q, N286T, L348V, | |
| GNA13 | I34T, G57S, S62F, M68K, Q134R, Y145F, L152F, | NM_006572 |
| | E167D, Q169H, R264H, E273K, V322G, V362G, | |
| | L371F | |
| GNAS | R201S, R201C, R201H, R201L, Q227K, Q227R, Q227L, | NM_000516 |
| | Q227H, R374C | |
| GNB1 | K57N, K57M, K57E, K57T, I80T, I80N | NM_002074 |
| IDH1 | R132C, R132G, R132H, R132L, R132P, R132V, V178I | NM_005896 |
| IDH2 | R140W, R140Q, R140L, R140G, R172W, R172G, | NM_002168 |
| | R172K, R172T, R172M, R172N, R172S | |
| IKZF1 | Frameshift/nonsense | NM_006060 |
| IKZF2 | Frameshift/nonsense | NM_016260 |
| IKZF3 | Frameshift/nonsense | NM_012481 |
| JAK1 | T478A, T478S, V623A, A634D, L653F, R724H, R724Q, | NM_002227 |
| | R724P, T782M, L783F | |
| JAK2 | N533D, N533Y, N533S, H538R, K539E, K539L, I540T, | NM_004972 |
| | I540V, V617F, R683S, R683G, del/ins537-539L, | |
| | del/ins538-539L, del/ins540-543MK, del/ins540- | |
| | 544MK, del/ins541-543K, del542-543, del543-544, | |
| | ins11546-547 | |
| JAK3 | M511T, M511I, A572V, A572T, A573V, R657Q, V715I, | NM_000215 |
| | V715A | |
| KDM6A | Frameshift/nonsense/splice-site, del419 | NM_021140 |
| KIT | ins503, V559A, V559D, V559G, V559I, V560D, V560A, | NM_000222 |
| | V560G, V560E, del560, E561K, del579, P627L, P627T, | |
| | R634W, K642E, K642Q, V654A, V654E, H697Y, | |
| | H697D, E761D, K807R, D816H, D816Y, D816F, | |
| | D816I, D816V, D816H, del551-559 | |
| KRAS | G12D, G12A, G12E, G12V, G13D, G13C, G13Y, | NM_033360 |
| | G13F, G13R, G13A, G13V, G13E, V14I, T58I, G60D, | |
| | G60A, G60V, Q61K, Q61E, Q61P, Q61R, Q61L, | |
| | Q61H, K117E, K117N, A146T, A146P, A146V | |
| LUC7L2 | Frameshift/nonsense/splice-site | NM_016019 |
| MLL | Frameshift/nonsense | NM_005933 |
| MLL2 | Frameshift/nonsense | NM_003482 |
| MPL | S505G, S505N, S505C, L510P, del513, W515A, W515R, | NM_005373 |
| | W515K, W515S, W515L, A519T, A519V, Y591D, | |
| | W515-518KT | |
| NF1 | Frameshift/nonsense | NM_000267 |
| NPM1 | Frameshift p.W288fs (insertion at c.859_860, 860_861, | NM_002520 |
| | 862_863, 863_864) | |
| NRAS | G12S, G12R, G12C, G12N, G12P, G12Y, G12D, | NM_002524 |
| | G12A, G12V, G12E, G13S, G13R, G13C, G13N, | |
| | G13P, G13Y, G13D, G13A, G13V, G13E, G60E, | |
| | G60R, Q61R, Q61L, Q61K, Q61P, Q61H, Q61Q | |
| PDS5B | Frameshift/nonsense/splice-site, R1292Q | NM_015032 |
| PDSS2 | Frameshift/nonsense | NM_020381 |
| PHF6 | Frameshift/nonsense/splice-site, A40D, M125I, S246Y, | NM_001015877 |
| | F263L, R274Q, C297Y, H302Y, H329L | |
| PHIP | Frameshift/nonsense/splice-site | NM_017934 |
| PPM1D | Frameshift/nonsense, exon 5 or 6 | NM_003620 |
| PRPF40B | Frameshift/nonsense/splice-site, P15H, M58I, P405L, | NM_001031698 |
| | P562S | |
| PRPF8 | M1307I, C1594W, D1598Y, D1598N, D1598V | NM_006445 |
| PTEN | Frameshift/nonsense/splice-site, D24G, R47G, F56V, | NM_000314 |
| | L57W, H61R, K66N, Y68H, C71Y, F81C, Y88C, D92G, | |
| | D92V, D92E, H93Y, H93D, H93Q, N94I, P95L, | |
| | I101T, C105F, C105S, D107Y, L112V, H123Y, C124R, | |
| | C124S, K125E, A126D, K128N, R130G, R130Q, | |
| | R130L, G132D, I135V, I135K, C136R, C136F, K144Q, | |
| | A151T, D153Y, D153N, Y155H, Y155C, R159K, | |
| | R159S, R161K, R161I, G165R, G165E, S170N, S170I, | |
| | R173C, Y174D, Y177C, H196Y, R234W, G251C, | |
| | D252Y, F271S, D326G | |
| PTPN11 | G60V, G60R, G60A, D61Y, D61V, D61G, Y63C, | NM_002834 |
| | E69K, E69G, E69D, E69Q, F71L, F71K, A72T, | |
| | A72V, A72D, T73I, E76K, E76Q, E76M, E76A, E76G, | |
| | E139G, E139D, N308D, N308T, N339S, P491L, S502P, | |
| | S502A, S502L, G503V, G503G, G503A, G503E, | |
| | Q506P, T507A, T507K | |
| RAD21 | Frameshift/nonsense/splice-site, R65Q, H208R, Q474R | NM_006265 |
| RUNX1 | Frameshift/nonsense/splice-site, S73F, H78Q, H78L, | NM_001001890 |
| | R80C, R80P, R80H, L85Q, P86L, P86H, S114L, D133Y, | |
| | L134P, R135G, R135K, R135S, R139Q, R142S, A165V, | |
| | R174Q, R177L, R177Q, A224T, D171G, D171V, | |
| | D171N, R205W, R223C | |
| SETBP1 | D868N, D868T, S869N, G870S, I871T, D880N, D880Q | NM_015559 |
| SETD2 | Frameshift/nonsense, V1190M | NM_014159 |
| SETDB1 | Frameshift/nonsense, K715E | NM_001145415 |
| SF1 | Frameshift/nonsense/splice-site, T454M, Y476C, | NM_004630 |
| | A508G | |
| SF3A1 | Frameshift/nonsense/splice-site, A57S, M117I, K166T, | NM_005877 |
| | Y271C | |
| SF3B1 | G347V, R387W, R387Q, E592K, E622D, Y623C, | NM_012433 |
| | R625L, R625C, R625G, H662Q, H662D, T663I, | |
| | K666N, K666T, K666E, K666R, K700E, V701F, | |
| | A708T, G740R, G740E, A744P, D781G, E783K, | |
| | R831Q, L833F, E862K, R957Q | |
| SRSF2 | Y44H, P95H, P95L, P95T, P95R, P95A, P107H, P95fs | NM_003016 |
| SMC1A | K190T, R586W, M689V, R807H, R1090H, R1090C | NM_006306 |
| SMC3 | Frameshift/nonsense, R155I, Q367E, D392V, K571R, | NM_005445 |
| | R661P, G662C | |
| STAG1 | Frameshift/nonsense/splice-site, H1085Y | NM_005862 |
| STAG2 | Frameshift/nonsense/splice-site | NM_006603 |
| SUZ12 | Frameshift/nonsense | NM_015355 |
| TET2 | Frameshift/nonsense/splice-site, missense mutations in | NM_001127208 |
| | catalytic domains (p.1104-1481 and 1843-2002) | |
| TP53 | Frameshift/nonsense/splice-site, S46F, G105C, G105R, | NM_001126112 |
| | G105D, G108S, G108C, R110L, R110C, T118A, T118R, | |
| | T118I, S127F, S127Y, L130V, L130F, K132Q, K132E, | |
| | K132W, K132R, K132M, K132N, F134V, F134L, | |
| | F134S, C135W, C135S, C135F, C135G, C135Y, Q136K, | |
| | Q136E, Q136P, Q136R, Q136L, Q136H, A138P, | |
| | A138V, A138A, A138T, T140I, C141R, C141G, C141A, | |
| | C141Y, C141S, C141F, C141W, V143M, V143A, | |
| | V143E, L145Q, W146C, W146L, L145R, V147G, | |
| | P151T, P151A, P151S, P151H, P151R, P152S, P152R, | |
| | P152L, T155P, T155A, V157F, R158H, R158L, A159V, | |
| | A159P, A159S, A159D, A161T, A161D, Y163N, | |
| | Y163H, Y163D, Y163S, Y163C, K164E, K164M, | |
| | K164N, K164P, H168Y, H168P, H168R, H168L, | |
| | H168Q, M169I, M169T, M169V, E171K, E171Q, | |
| | E171G, E171A, E171V, E171D, V172D, V173M, | |
| | V173L, V173G, R174W, R175G, R175C, R175H, | |
| | C176R, C176G, C176Y, C176F, C176S, P177R, P177R, | |
| | P177L, H178D, H178P, H178Q, H179Y, H179R, | |
| | H179Q, R181C, R181Y, D186G, G187S, P190L, P190T, | |
| | H193N, H193P, H193L, H193R, L194F, L194R, I195F, | |
| | I195N, I195T, R196P, V197L, G199V, Y205N, Y205C, | |
| | Y205H, D208V, R213Q, R213P, R213L, R213Q, | |
| | H214D, H214R, S215G, S215I, S215R, V216M, V217G, | |
| | Y220N, Y220H, Y220S, Y220C, E224D, I232F, I232N, | |
| | I232T, I232S, Y234N, Y234H, Y234S, Y234C, Y236N, | |
| | Y236H, Y236C, M237V, M237K, M237I, C238R, | |
| | C238G, C238Y, C238W, N239T, N239S, S241Y, S241C, | |
| | S241F, C242G, C242Y, C242S, C242F, G244S, G244C, | |
| | G244D, G245S, G245R, G245C, G245D, G245A, | |
| | G245V, G245S, M246V, M246K, M246R, M246I, | |
| | N247I, R248W, R248G, R248Q, R249G, R249W, | |
| | R249T, R249M, P250L, I251N, L252P, I254S, I255F, | |
| | I255N, I255S, L257Q, L257P, E258K, E258Q, D259Y, | |
| | S261T, G262D, G262V, L265P, G266R, G266E, | |
| | G266V, R267W, R267Q, R267P, E271K, V272M, | |
| | V272L, R273S, R273G, R273C, R273H, R273P, R273L, | |
| | V274F, V274D, V274A, V274G, V274L, C275Y, C275S, | |
| | C275F, A276P, C277F, C277Y, P278T, P278A, P278S, | |
| | P278H, P278R, P278L, G279E, R280G, R280K, R280T, | |
| | R280I, R280S, D281N, D281H, D281Y, D281G, | |
| | D281E, R282G, R282W, R282Q, R282P, E285K, | |
| | E285V, E286G, E286V, E286K, K320N, L330R, | |
| | G334V, R337C, R337L, A347T, L348F, T377P | |
| U2AF1 | D14G, S34F, S34Y, R35L, R156H, R156Q, Q157R, | NM_006758 |
| | Q157P | |
| U2AF2 | R18W, Q143L, M144I, L187V, Q190L | NM_007279 |
| WT1 | Frameshift/nonsense/splice-site | NM_024426 |
| ZRSR2 | Frameshift/nonsense, R126P, E133G, C181F, H191Y, | NM_005089 |
| | I202N, F239V, F239Y, N261Y, C280R, C302R, C326R, | |
| | H330R, N382K | |

F. Patient Profiles

In addition to having one or more loss-of-function mutations in the genes DNMT3A, TET2, JAK2, and/or ASXL1, a human subject benefitting herein may have one or more of the following patient profile characteristics. For example, the human subject may also exhibit one or more of the following risk factors: increased levels of cholesterol, increased levels of liver iron, increased alcohol consumption, increased body mass index, and obesity.

In some embodiments, increased levels of cholesterol are cholesterol amounts in the blood that are higher than the recommended range of cholesterol for one's age. In some embodiments, increased levels of cholesterol are cholesterol amounts in the blood that are higher than normal based on one's medical history. In some embodiments, increased levels of cholesterol may increase one's risk for CHIP, and/or chronic liver diseases, such as liver fibrosis and cirrhosis. In some embodiments, increased levels of cholesterol may cause no symptoms or may not present as a risk factor. In one embodiment, high cholesterol levels mean at least 200 mg/dL of total cholesterol and/or at least 100 mg/dL of LDL and/or at least 130 mg/dL of non-HDL cholesterol.

In some embodiments, increased levels of liver iron are iron amounts in the liver that are higher than the normal range as determined by blood test, magnetic resonance imaging (MRI), and/or liver biopsy. In some embodiments, the amount of iron in the liver is greater than 70 μmol iron/g-dry weight. In some embodiments, increased levels of liver iron comprise increased levels of blood iron. In some embodiments, increased levels of liver iron may increase one's risk for CHIP, and/or chronic liver diseases, such as liver fibrosis and cirrhosis. In some embodiments, increased levels of liver iron may cause no symptoms or may not present as a risk factor.

The Office of Disease Prevention and Health Promotion of the US Department of Health and Human Services defines moderate alcohol consumption as one or two drinks per day, wherein one alcoholic drink-equivalent may be described as containing 14 g (0.6 fl oz) of pure alcohol. In some embodiments, increased alcohol consumption may be more than one or two drinks per day. In some embodiments, increased alcohol consumption may occur on a single occasion or over time. In some embodiments, increased alcohol consumption comprises binge drinking. In some embodiments, increased alcohol consumption may increase one's risk for CHIP, and/or chronic liver diseases, such as liver fibrosis and cirrhosis.

According to the Centers for Disease Control and Prevention, body mass index (BMI) is a person's weight in kilograms divided by the square of height in meters. In some embodiments, BMI may be used to measure obesity and body fatness. In some embodiments, such as in children and teenagers, age and sex are taken into consideration when determining BMI. In some embodiments, such as in adults, age and sex may not be taken into consideration when determining BMI. In some embodiments, such as in athletes, a high BMI because of increased muscularity may not be an indicator of obesity or body fatness. In some embodiments, increased body mass index may increase one's risk for CHIP, and/or chronic liver diseases, such as liver fibrosis and cirrhosis. Obesity may be defined as a BMI of 30.0 or higher. In some embodiments, obesity may increase one's risk for CHIP, and/or chronic liver diseases, such as liver fibrosis and cirrhosis.

G. Therapies

Pharmaceutical agents and lifestyle modifications may be employed with the methods described in this application. In many embodiments, the mutations and increased risk factors described herein may lead to undesirable symptoms, signs, causes, or increased risk for liver disease. These therapeutic approaches may provide a benefit to human subjects with liver disease. In some embodiments, the method may include (a) administering an effective amount of pharmaceutical agent, and/or (b) prescribing a lifestyle modification.

1. Pharmaceutical Agents

In some embodiments, the pharmaceutical agent for treating liver disease targets NLRP3 inflammasome, IL-1β, IL-6, IL-6 receptor, CCL22, MCP1, and/or MIP2. In some embodiments, the pharmaceutical agent is an inhibitor of NLRP3 inflammasome, IL-1β, IL-6, IL-6 receptor, CCL22, MCP1, and/or MIP2. In other embodiments, the pharmaceutical agent is an antibody or an antigen binding fragment that binds to NLRP3 inflammasome, IL-1β, IL-6, IL-6 receptor, CCL22, MCP1, and/or MIP2. In some embodiments, the pharmaceutical agent is an antibody or an antigen binding fragment thereof.

a) NLRP3 Inflammasome

Various approaches to inhibiting NLRP3 inflammasome may be employed. NLRP3 is an intracellular sensor that detects a broad range of microbial motifs, endogenous signaling molecules and environmental irritants, resulting in the formation and activation of the NLRP3 inflammasome. Assembly of the NLRP3 inflammasome leads to caspase 1-dependent release of cytokines IL-1β, IL-6, and IL-18, as well as to gasdermin D-mediated pyroptotic cell death. Treatment with a select NLRP3 inflammasome inhibitor has been shown to protect against development of Tet2-mediated clonal hematopoiesis heart failure. Sano et al., Tet2-mediated clonal hematopoiesis accelerates heart failure through a mechanism involving the IL-1β/NLRP3 inflammasome, J. Am. Coll. Cardiol. 71:875-886 (2018). In some embodiments, inhibition of NLRP3 inflammasome may provide a benefit to human subjects with liver disease.

NLRP3 inflammasome activity may be reduced using an NLRP3 inflammasome depleting drug or an NLRP3 inflammasome activity reducing drug. For instance, the NLRP3 inflammasome inhibitor may comprise an anti-NLRP3 inflammasome antibody or antigen binding fragment thereof. In some embodiments, the NLRP3 inflammasome inhibitor comprises melatonin and/or methylprednisolone.

In some embodiments, the human subject does not have a mutation in NLRMP3 that inhibits the NLRMP3/IL-1β/IL-6 signaling blockade.

b) IL-1β

Various approaches to inhibiting IL-1β may be employed. TET2 deficiency in hematopoietic cells is associated with greater cardiac dysfunction in murine models of heart failure due to elevated IL-1β signaling. Sano et al., Tet2-mediated clonal hematopoiesis accelerates heart failure through a mechanism involving the IL-1β/NLRP3 inflammasome, J. Am. Coll. Cardiol. 71:875-886 (2018). In some embodiments, inhibition of IL-1β may provide a benefit to human subjects with liver disease.

IL-1β activity may be reduced using an IL-1β depleting drug or an IL-1β activity reducing drug. For instance, the IL-1β inhibitor may comprise an anti-IL-1β antibody or antigen binding fragment thereof. In some embodiments, the IL-1β inhibitor comprises AC-201, canakinumab (Ilaris®), and/or anakinra (Kineret®).

In some embodiments, the human subject does not have a mutation in IL-1β that inhibits the NLRMP3/IL-1β/IL-6 signaling blockade.

c) IL-6

Various approaches to inhibiting IL-6 may be employed. IL-6 deficiency has been shown to attenuate cardiovascular risk in clonal hematopoiesis. Bick et al., Genetic interleukin 6 signaling deficiency attenuates cardiovascular risk in clonal hematopoiesis, Circulation 141:124-131 (2020). In some embodiments, inhibition of IL-6 may provide a benefit to human subjects with liver disease.

IL-6 activity may be reduced using an IL-6 depleting drug or an IL-6 activity reducing drug. For instance, the IL-6 inhibitor may comprise an anti-IL-6 antibody or antigen binding fragment thereof. In some embodiments, the IL-6 inhibitor comprises clazakizumab and/or ziltivekimab.

In some embodiments, the human subject does not have a mutation in IL-6 that inhibits the NLRP3/IL-1β/IL-6 signaling blockade. In some embodiments, does not have a D358A mutation in IL6R.

d) IL-6 Receptor

Various approaches to inhibiting the IL-6 receptor may be employed. Deficiency in IL-6 signaling has been shown to attenuate cardiovascular risk in clonal hematopoiesis. Bick et al., Genetic interleukin 6 signaling deficiency attenuates cardiovascular risk in clonal hematopoiesis, Circulation 141:124-131 (2020). In some embodiments, inhibition of IL-6 receptor may provide a benefit to human subjects with liver disease.

IL-6 receptor activity may be reduced using an IL-6 receptor depleting drug or an IL-6 receptor activity reducing drug. For instance, the IL-6 receptor inhibitor may comprise an anti-IL-6 receptor antibody or antigen binding fragment thereof. In some embodiments, the IL-6 receptor inhibitor comprises tocilizumab and/or sarilumab.

In some embodiments, the human subject does not have a mutation in IL-6 receptor that inhibits the NLRP3/IL-1β/IL-6 signaling blockade.

e) CCL22

Various approaches to inhibiting CCL22 may be employed. Transcriptional and protein levels of CCL22 are elevated in TET2^{-/-} transplanted mice. In some embodiments, inhibition of CCL22 may provide a benefit to human subjects with liver disease.

CCL22 activity may be reduced using a CCL22 depleting drug or a CCL22 activity reducing drug. For instance, the CCL22 inhibitor may comprise an anti-CCL22 antibody or antigen binding fragment thereof. In some embodiments, the CCL22 inhibitor comprises sinomenine and/or fucoidan.

In some embodiments, the human subject does not have a mutation in CCL22 that decreases CCL22 expression or activity.

f) MCP1

Various approaches to inhibiting MCP1 may be employed. MCP1 protein levels are elevated in cultured liver slices from Tet2^{-/-} transplanted mice. In some embodiments, inhibition of MCP1 may provide a benefit to human subjects with liver disease.

MCP1 activity may be reduced using an MCP1 depleting drug or an MCP1 activity reducing drug. For instance, the MCP1 inhibitor may comprise an anti-MCP1 antibody or antigen binding fragment thereof. In some embodiments, the MCP1 inhibitor comprises bindarit.

In some embodiments, the human subject does not have a mutation in MCP1 that decreases MCP1 expression or activity.

g) MIP2

Various approaches to inhibiting MIP2 may be employed. MIP2 protein levels are elevated in cultured liver slices from Tet2^{-/-} transplanted mice. In some embodiments, inhibition of MIP2 may provide a benefit to human subjects with liver disease.

MIP2 activity may be reduced using an MIP2 depleting drug or an MIP2 activity reducing drug. For instance, the MIP2 inhibitor may comprise an anti-MIP2 antibody or antigen binding fragment thereof.

In some embodiments, the human subject does not have a mutation in MIP2 that decreases MIP2 expression or activity.

h) Hepatitis Treatment

Various approaches to treating hepatitis may be employed. Treatment for hepatitis varies, depending on the type and severity of the disease. Hepatitis is an infection that causes liver inflammation that can progress to fibrosis (scarring), cirrhosis or liver cancer. Hepatitis may be caused by one of five main hepatitis viruses, referred to as types A, B, C, D, and E. Hepatitis may be treated using an antiviral drug or a vaccine. In some embodiments, the hepatitis treatment comprises sofosbuvir, ribavirin, pegylated interferon, interferon alpha, daclatasvir, adefovir, entecavir, lamivudine, telbivudine, tenofovir, elbasvir-grazoprevir, ledipasvir-sofosbuvir, sofosbuvir-velpatasvir, ombitasvir-paritaprevir-ritonavir, ombitasvir-paritaprevir-ritonavir-dasabuvir, and/or a vaccine.

i) Cirrhosis Treatment

Various approaches to preventing or treating cirrhosis may be employed to slow down the progression of scar tissue in the liver and to prevent or treat symptoms and complications or cirrhosis. In some embodiments, the cirrhosis treatment comprises a treatment to prevent ascites, edema, portal hypertension, severe bleeding, or infections.

2. Lifestyle Modifications

Lifestyle modifications may also be used as liver disease prevention or treatment. One or more lifestyle modifications may be prescribed to a human subject in combination with a pharmaceutical agent described herein. At least one lifestyle modification may be chosen from weight loss, exercise, and dietary modification. In some embodiments, dietary modification may be chosen from cessation or reduction of alcohol consumption, a low sodium diet, a low fat diet, a low carbohydrate diet, a high fiber diet, and cessation or reduction of medications and/or supplements that are toxic to the liver.

### II. Diagnostic Methods

The methods of the invention include diagnostic applications. In some embodiments, a method for diagnosing liver disease, a method for diagnosing CHIP, a method for predicting risk for CHIP, and/or a method for targeted prevention of liver disease in a human subject comprises (a) obtaining a nucleic acid sample from the subject; (b) detecting whether the sample contains at least one DNMT3A, TET2, JAK2, and/or ASXL1 mutation; and (c) diagnosing the subject as having liver disease, CHIP, a risk for CHIP, and/or risk of liver disease, respectively, when at least one DNMT3A, TET2, JAK2, and/or ASXL1 mutation is detected. While not being bound by theory, the methods are presumed to be related by diagnosing CHIP or a risk for CHIP, which can thereby facilitate liver disease risk prediction and targeted prevention, as well as diagnosis of liver disease itself.

In some embodiments, the nucleic acid sample is obtained from a blood sample.

Various technologies may be employed to detect the one or more mutations disclosed in the current application. In many embodiments, the one or more mutations are detected by a DNA or RNA sequencing method. In many embodiments, the one or more mutations are detected by DNA microarray. In some embodiments, the one or more mutations are detected by a single-nucleotide-polymorphism (SNP) array.

In some embodiments, method further comprises a DNA sequencing method to detect whether the sample contains at least one DNMT3A, TET2, JAK2, and/or ASXL1 mutation. In some embodiments, the sequencing is carried out with one or more cells in a blood sample of the human subject.

In some embodiments, the method further comprises determining the presence or absence of mutations in a human subject. In some embodiments, a loss-of-function mutation in DNMT3A, TET2, JAK2, and/or ASXL1. In some embodiments, the method further comprises determining an amino acid change in TET2 chosen from S145N, S282F, A308T, N312S, L346P, P399L, S460F, D666G, S817T, P941S, C1135Y, R1167T, I1175V, S1204C, R1214W, D1242R, D1242V, Y1245S, R1261C, R1261H, R1261L, F1287L, W1291R, K1299E, K1299N, R1302G, E1318G, P1367S, C1396W, L1398R, V1417F, G1869W, L1872P, I1873T, C1875R, H1881Q, H1881R, R1896M, R1896S, S1898F, V1900A, G1913D, A1919V, R1926H, P1941S, P1962L, R1966H, R1974M, and R2000K. In some embodiments, a TP52, S3FB1, SRSF2, GNB1, CBL, and/or PPM1D mutation is also detected in the human subject.

In some embodiments, the human subject is determined not to have a mutation in NLRP3, IL-1β, and/or IL-6 that inhibits the NLRP3/IL-1β/IL-6 signaling blockade. In some embodiments, the human subject is determined not to have a D358A mutation in IL6R.

In some embodiments, the method further comprises determining if the human subject exhibits one or more risk factors chosen from increased levels of cholesterol, increased levels of liver iron, increased alcohol consumption, increase body mass index, and/or obesity.

In some embodiments, the method further comprises determining if the human subject does not exhibit other conditions such as myeloproliferative neoplasm and portal vein thrombosis.

Diagnosis, including all of the aspects described herein, may be used alone or in combination with therapy, as described herein.

### III. Definitions

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition, which includes a prophylactic treatment. The term “therapeutically effective amount” can encompass an amount that improves overall therapy, reduces or avoids symptoms, signs, or causes of the condition, and/or enhances the therapeutic efficacy of another therapeutic agent.

The term “diagnosing” refers to identifying an increased risk of the disease.

### EXAMPLES

### Example 1a: Association of CHIP with Prevalent and Incident Chronic Liver Disease

A. Methods

1. Study Samples

We examined the association between CHIP and chronic liver disease using five datasets. For analysis of prevalent disease, we used data from the Framingham Heart Study (FHS, n=4114), Atherosclerosis Risk in Communities study (ARIC, n=7414) and a subsample of UK Biobank who underwent whole exome sequencing (WES, n=46,714, Table 2).

TABLE 2
Chronic liver diseases cases and definitions in five samples

| Cohort | Cases | Controls | Definition | Analysis |
|---|---|---|---|---|
| Framingham Heart Study | 36 | 4194 | Prevalent chronic liver | Observational |
| | | | disease, physician- | |
| | | | diagnosed | |
| Atherosclerosis Risk in | 65 | 8457 | Prevalent cirrhosis or | Observational |
| Communities Study | | | chronic liver disease, | |
| | | | physician-diagnosed | |
| UK Biobank WES | 39 | 46674 | Prevalent cirrhosis, | Observational |
| | | | hospitalization or death | |
| | | | due to ICD codes K70.2, | |
| | | | K70.3, K70.4, K74.0, | |
| | | | K74.1, K74.2, K74.6, | |
| | | | K76.6, or I85 | |
| UK Biobank Array | 1170 | 361913 | Incident cirrhosis, | Observational |
| | | | hospitalization or death | |
| | | | due to ICD codes K70.2, | |
| | | | K70.3, K70.4, K74.0, | |
| | | | K74.1, K74.2, K74.6, | |
| | | | K76.6, or I85 | |
| Multi-cohort GWAS (Emdin | 5777 | 487780 | Prevalent cirrhosis, | Mendelian |
| et al., A missense variant in | | | hospitalization or death | randomization |
| Mitochondrial Amidoxime | | | due to cirrhosis | |
| Reducing Component 1 gene | | | | |
| and protection against liver | | | | |
| disease, bioRxiv, | | | | |
| doi:doi.org/10.1101/594523 | | | | |
| (2019). | | | | |

Individuals in UK Biobank who underwent WES were selected to enrich for participants who had magnetic resonance imaging, linked hospitalization records and linked primary care records. Van Hout et al., Whole exome sequencing and characterization of coding variation in 49,960 individuals in the UK Biobank, bioRxiv, doi:doi.org/10.1101/572347 (2019). For incident chronic liver disease, we tested the association of genotyped CHIP variants with chronic liver disease in a separate sample of 363,083 individuals from UK Biobank. Individuals with prevalent leukemia or other hematologic malignancy were excluded from analysis.

We defined chronic liver disease as the development of liver fibrosis or cirrhosis, combining the following ICD10 diagnostic codes: K70.2 (alcoholic fibrosis and sclerosis of the liver), K70.3 (alcoholic cirrhosis of the liver), K70.4 (alcoholic hepatic failure), K74.0 (hepatic fibrosis), K74.1 (hepatic sclerosis), K74.2 (hepatic fibrosis with hepatic sclerosis), K74.6 (other and unspecified cirrhosis of liver), K76.6 (portal hypertension), or I85 (esophageal varices). These ICD codes have previously been demonstrated to have high specificity for identifying patients with cirrhosis compared to physician review and associate strongly with known cirrhosis loci. Emdin et al., A missense variant in Mitochondrial Amidoxime Reducing Component 1 gene and protection against liver disease, bioRxiv, doi:doi.org/10.1101/594523 (2019). Alcohol-related liver disease was defined as chronic liver disease among individuals with excess alcohol intake (≥21 drinks per week for men or ≥14 drinks per week for women) and no history of hepatitis B or hepatitis C. Chalasani et al., The diagnosis and management of nonalcoholic fatty liver disease: Practice guidance from the American Association for the Study of Liver Diseases. Hepatology 67:328-357 (2018). Non-alcoholic steatohepatitis (NASH) was defined as chronic liver disease among individuals consuming less than 21 drinks per week for men and less than 14 drinks per week for women and no history of hepatitis B or hepatitis C as outlined in the American Association for the Study of Liver Diseases guidelines. Chalasani et al., The diagnosis and management of nonalcoholic fatty liver disease: Practice guidance from the American Association for the Study of Liver Diseases. Hepatology 67:328-357 (2018).

2. Whole-Exome Sequencing and CHIP Ascertainment

We identified individuals with CHIP based on a prespecified list of variants in 74 genes recurrently mutated in myeloid cancers (Table 1). For prevalent liver disease analyses, CHIP was ascertained using WES. Bick et al., Genetic interleukin 6 signaling deficiency attenuates cardiovascular risk in clonal hematopoiesis, Circulation 141:124-131 (2020); Bick et al., Inherited causes of clonal hematopoiesis of indeterminate potential in TOPMed whole genomes, bioRxiv doi:doi.org/10.1101/782748 (2019). For incident liver disease analysis, CHIP was ascertained using array-derived genotype variants in AXSL1, DNMT3A, JAK2, and TET2 (Table 3).

TABLE 3
CHIP variants ascertained through genotyping in the UK biobank

| | | | | Reference | Effect | |
|---|---|---|---|---|---|---|
| Variant | Gene | Chr | Location | Allele | Allele | Consequence |
| rs373145711 | ASXL1 | 20 | 31021211 | C | T | Arg404Ter |
| rs373873045 | DNMT3A | 2 | 25459834 | C | A | Glu817Ter |
| rs765045799 | DNMT3A | 2 | 25462086 | T | C | Splice Acceptor |
| rs1190050788 | DNMT3A | 2 | 25466765 | A | C | Splice Donor |
| rs568207978 | DNMT3A | 2 | 25467083 | G | A | Arg598Ter |
| rs369109129 | DNMT3A | 2 | 25469922 | G | A | Gln374Ter |
| rs776841024 | DNMT3A | 2 | 25470556 | C | T | Trp306Ter |
| rs767439400 | DNMT3A | 2 | 25471030 | GGGCT | G | SerPro243Fs |
| rs77375493 | JAK2 | 9 | 5073770 | G | T | Val617Phe |
| rs370735654 | TET2 | 4 | 106196213 | C | T | Arg1516Ter |
| rs757144251 | TET2 | 4 | 106157271 | TA | T | Lys725Fs |

We examined genotyping fidelity of each variant by manually examining imaging files with ScatterShot. Individuals included in WES and prevalence analysis were excluded from this analysis.

B. Results

We examined whether CHIP is associated with elevated risk of chronic liver disease in three cohorts. We tested the association of CHIP with prevalent liver disease using data from the Framingham Heart Study (FHS, n=4114), Atherosclerosis Risk in Communities study (ARIC, n=7414) and a subsample of UK Biobank who underwent whole exome sequencing (n=46,714, Table 2). In these cohorts, the mean (standard deviation) age range was 57-61 (6-16) and the prevalence of CHIP varied between 4% and 7% (Table 4).

TABLE 4
Baseline characteristics of participants in samples analyzed

| | | | | | UK Biobank | | UK Biobank | |
|---|---|---|---|---|---|---|---|---|
| | FHS, | | ARIC, | | WES, | | Array, | |
| Characteristics | n = 4230 | | n = 8522 | | n = 46713 | | n = 363083 | |
| Age, mean years (SD) | 38.3 | (10.1) | 57.4 | (6.0) | 57.2 | (8.0) | 56.9 | (8.1) |
| Female Gender, n (%) | 2228 | (54%) | 4358 | (56%) | 21380 | (45.7) | 195403 | (54%) |
| BMI, mean kg/m² (SD) | 27.8 | (5.5) | 27.7 | (5.4) | 27.4 | (4.8) | 27.4 | (4.8) |
| Current smoking, n (%) | 610 | (15%) | 1992 | (26%) | 2975 | (6%) | 29160 | (8%) |
| Alcohol intake, mean | 6.8 | (16.6) | 5.6 | (8.8) | 7.8 | (9.7) | 8.1 | (10.1) |
| drinks per week (SD) | | | | | | | | |
| History of diabetes | 162 | (4.0%) | 674 | (9%) | 2436 | (5%) | 19141 | (5%) |
| mellitus, n (%) | | | | | | | | |
| CHIP prevalence, n (%) | 247 | (6%) | 333 | (4%) | 3086 | (7%) | 266 | (0.1%) |

FHS, Framingham Heart Study; ARIC, Atherosclerosis Risk in Communities study; BMI, body-mass index; SD, standard deviation; WES, whole exome sequencing.

DNMT3A and TET2 were the most commonly mutated gene (40%). The prevalence of CHIP increases across cohorts with increasing age. Known associations with CHIP, including age, sex, type 2 diabetes, smoking and self-reported ethnicity exhibited similar associations to CHIP across cohorts (Table 2).

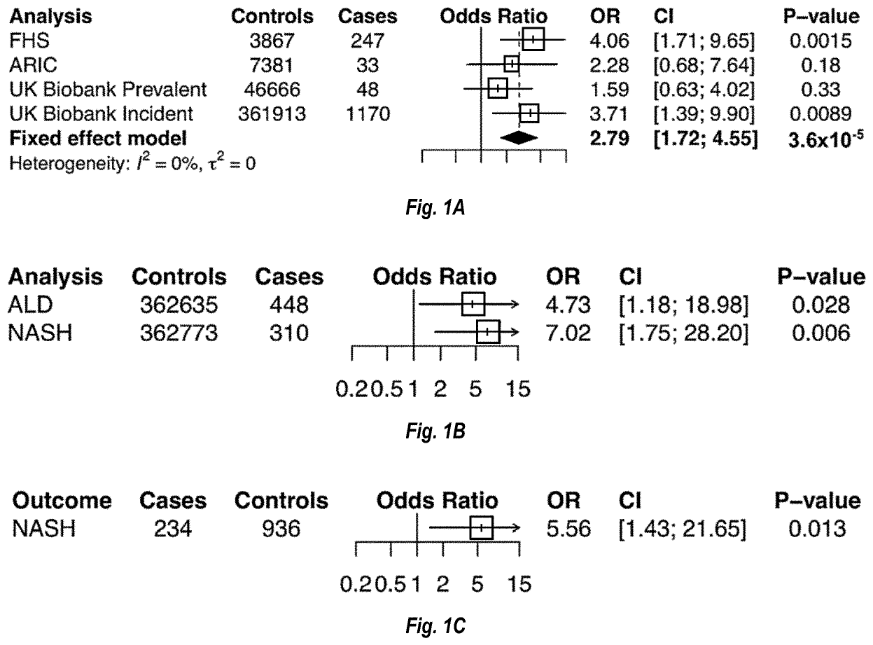

In FHS, individuals with CHIP were at four-fold odds of having chronic liver disease (OR 4.06 CI 1.71, 9.65, FIGS. 1A-C). In ARIC and UK Biobank, individuals with CHIP consistently demonstrated increased risk for chronic liver disease at baseline (OR 2.28 CI 0.68, 7.64 and OR 1.59 CI 0.63, 4.02, respectively) (FIGS. 1A-C). Overall CHIP was associated with a 2.6-fold risk of prevalent chronic liver disease (OR 2.55 CI 1.45, 4.46, p<0.001). CHIP with variant allele fraction (VAF)>10%, previously linked to excess non-hepatological outcomes, was associated with a greater risk of chronic liver disease than CHIP with VAF<10% (OR 2.47 vs 1.65, p_{interaction}=0.46). Although JAK2 CHIP was associated with a highly elevated risk of chronic liver disease (OR 29.2 CI 3.9, 221, p=0.001), possibly due to prothrombotic effects, non-JAK2 CHIP was also associated with elevated risk of chronic liver disease (OR 2.29 CI 1.37, 3.85, p=0.002). TET2 CHIP was independently associated with a highly elevated risk of chronic liver disease (OR 5.6 CI 2.8, 11.4, p=2×10⁻⁶). Autosomal chromosomal mosaicism of blood cells was not associated with risk of chronic liver disease in UK Biobank (OR 1.18 CI 0.97, 1.45, p=0.10). Loh et al., Insights into clonal haematopoiesis from 8,342 mosaic chromosomal alterations, Nature 559:350-355 (2018).

In an analysis of incident cirrhosis in UK Biobank, 1170 cases of incident chronic liver disease were observed among 363083 individuals who underwent genotyping with an array. 269 individuals carried a CHIP variant in DNMT3A, ASXL1, JAK2 or TET2 which was genotyped on the UK Biobank array. CHIP variants ascertained through genotyping had high positive predictive value for exome sequencing-ascertained CHIP (90%), had similar association with increasing age as exome sequencing-ascertained CHIP (OR 1.04 per year, p<0.001) and were strongly associated with incident myeloid hematologic malignancy (HR 106, CI 72, 158, p>0.001). Individuals with mutations in DNMT3A, ASXL1, JAK2 or TET2 were at nearly four-fold risk for developing incident liver disease (HR 3.71, 1.39, 9.9, p=0.009; FIGS. 1A-C) than non-carriers. Overall, presence of CHIP was associated with a nearly three-fold risk of prevalent or incident chronic liver disease (OR 2.88 CI 1.77, 4.69, p<0.001; FIGS. 1A-C). no evidence of heterogeneity was observed between estimates among cohorts or estimates between prevalent and incident chronic liver disease. Individuals with CHIP continued to be at elevated risk of chronic liver disease when baseline alcohol consumption, body mass index, alanine transaminase levels, aspartate transaminase levels and alkaline phosphatase levels were adjusted for (OR 4.00 CI 1.59, 9.99, p=0.003).

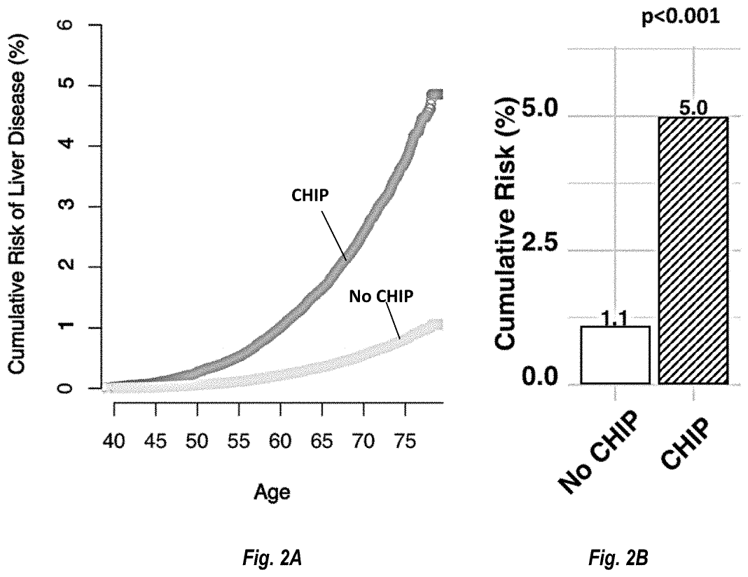

We examined cumulative risk of chronic liver disease in UK Biobank by array-derived CHIP status. Individuals without CHIP had a 1% cumulative incidence of chronic liver disease by age 80 years. In contrast, individuals with CHIP had a 5% cumulative incidence of liver disease (p<0.001, FIGS. 2A-B). For comparison, individuals with morbid obesity (BMI>35 kg/m²) had a 2.5% cumulative incidence of liver disease.

### Example 1b: Association of CHIP with Subtypes of Chronic Liver Disease

A. Methods

1. Biopsy Proven NASH Analysis

As described in Example 1a above, analyses of chronic liver disease were based primarily on ICD codes, we also examined whether CHIP predisposes to biopsy-proven non-alcoholic steatohepatitis (NASH). For this analysis, we analyzed 234 biopsy-proven NASH samples from Massachusetts General Brigham biobank. We matched them to 936 controls using age and sex. CHIP was ascertained through whole exome sequencing of blood samples deposited at enrollment in the MGB Biobank.

B. Results

When the subtypes of liver disease were examined, individuals with CHIP were at similarly elevated risk of both alcohol-related liver disease (ALD) and NASH (FIG. 1B). Only seven individuals with virus-related chronic liver disease (chronic liver disease and a history of hepatitis C or hepatitis B) could be identified, preventing ascertainment of the association of CHIP with chronic liver disease from viral causes.

To confirm the association of CHIP with NASH, we identified 234 biopsy-proven NASH cases in the Mass General Brigham Biobank. We matched them on age and sex to 936 controls. We performed exome sequencing and CHIP calling as previously described. NASH carriers were five times as likely to have a history of CHIP as matched controls (OR 5.56 CI 1.43, 21.65, p=0.01, FIG. 1C).

### Example 2: Association of CHIP with Liver Imaging and Biomarkers

A. Methods

1. Study Samples

To examine whether CHIP associates with serum biomarkers or liver imaging, we used data from UK Biobank. Blood samples were collected from all UK Biobank participants during their initial enrollment visit. These samples were used for both whole exome sequencing and for biomarker assays. In a cross-sectional analysis, we tested whether carrying CHIP associated with serum liver enzyme levels (alanine transaminase levels, aspartate transaminase levels, alkaline phosphatase levels, gamma glutamyl transferase levels) and serum inflammatory biomarkers (C-reactive protein, platelet count, hemoglobin and white blood cell count). Serum liver enzyme levels and C-reactive protein were measured by immunoassay using a Beckman Coulter Au5800. Blood cell counts were measured using a Beckman Coulter LH 750.

We examined whether CHIP status associated with liver fat and liver iron in 4434 individuals in UK Biobank with whole exome sequence data who underwent liver magnetic resonance imaging of their liver. Liver fat was measured using proton density fat fraction. Liver inflammation and fibrosis was measured using iron corrected T1 relaxation time (cT1). Banerjee et al., Multiparametric magnetic resonance for the non-invasive diagnosis of liver disease, Journal of Hepatology 60(1):69-77 (2014).

B. Results

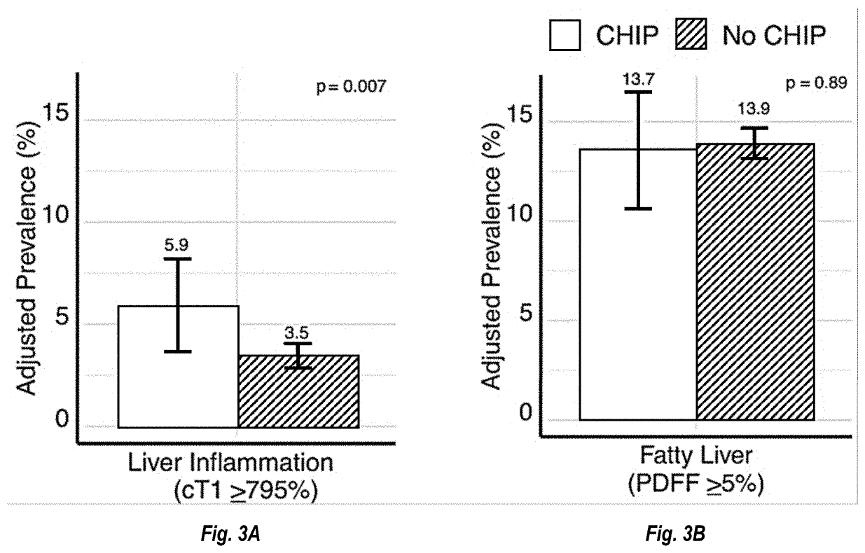

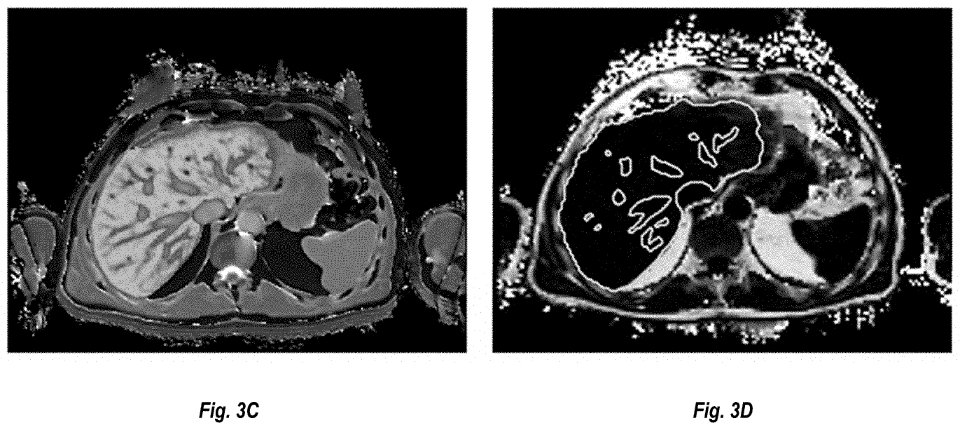

We examined whether CHIP may be associated with increased liver inflammation and liver fat using magnetic resonance imaging data from 8251 individuals in UK Biobank. CHIP was indeed associated with increased liver inflammation (proton cT1>795 ms, OR 1.74 CI 1.16, 2.60, p=0.007, FIGS. 3A and 3B). However, CHIP was not associated with hepatic steatosis (proton density fat fraction≥5%, OR 0.98 CI 0.75, 1.28, p=0.89, FIGS. 3A and 3C).

Using data from UK Biobank, we examined the relationship between CHIP and serum biomarkers in up to 393128 individuals. CHIP was not associated with alanine transaminase levels or aspartate transaminase levels. However, CHIP was associated with a modest increase in alkaline phosphatase levels (0.015 U/L, p=0.003), total bilirubin levels (0.31 m/dl, p=0.0001) and C-reactive protein levels (0.19 mg/dl, p=0.03). CHIP was also associated with a modest elevation in platelet count (8.5 per uL, p<0.001) and leukocyte count (0.11×10⁹ cells/L, p=0.01). When JAK2 CHIP carriers were excluded, CHIP continued to be associated with elevations in alkaline phosphatase levels, total bilirubin levels and C-reactive protein, but was not associated with platelet count.

### Example 3a: Mendelian Randomization Analysis of the Association of CHIP with Chronic Liver Disease

A. Methods

1. Statistical Analysis

We tested for the association of CHIP status with prevalent chronic liver disease using logistic regression, with adjustment for age, sex, type 2 diabetes and smoking. Additional analyses were performed after adjustment for alcohol consumption and body mass index. For incident chronic liver disease, we used Cox proportional hazards regression, with adjustment for age, sex, type 2 diabetes and smoking. Further adjustment for alcohol consumption and body mass index was performed. To test the association of CHIP status with liver fat (proton density fat fraction>5%) and liver inflammation and fibrosis (proton cT1>795 ms), we used logistic regression with adjustment for age and sex. We used Mendelian randomization analysis with robust adjusted profile score (MR-RAPS) to examine the association of genetic predisposition to CHIP with cirrhosis risk, which provides for control of type 1 error rate when using sub-genome wide significant genetic variants. Zhao et al., Statistical inference in two-sample summary-data Mendelian randomization using robust adjusted profile score, arXiv:1801.09652 (2018). All statistical analyses were conducted using R version 3.5 or Graphpad Prism 8.3.1.

To examine whether done size may modify the association of CHIP status with liver disease, as has been reported for hematologic malignancy and cardiovascular disease, we stratified patients by variant allele fraction≥0.1 and <0.1 and mutated gene. Bick et al., Genetic interleukin 6 signaling deficiency attenuates cardiovascular risk in clonal hematopoiesis, Circulation 141:124-131 (2020); Jaiswal et al., Age-related clonal hematopoiesis associated with adverse outcomes, N. Engl. J. Med. 371L2488-2498 (2014); Xie et al., Age-related mutations associated with clonal hematopoietic expansion and malignancies, Nat. Med. 20:1472-1478 (2014); Jaiswal et al., Clonal hematopoiesis and risk of atherosclerotic cardiovascular disease, N. Engl. J. Med. 377:111-121 (2017). We tested for the association of CHIP Status with liver fat (proton density fat fraction) and liver inflammation and fibrosis (proton cT1). We used logistic regression with adjustment for age and sex. Fatty liver was defined as fat fraction≥5%. Fibrotic liver was defined as proton cT1≥795 ms. Banerjee et al., Multiparametric magnetic resonance for the non-invasive diagnosis of liver disease, Journal of Hepatology 60(1):69-77 (2014).

To assess the causality of the association of CHIP status with chronic liver disease, we performed a Mendelian randomization analysis. We examined the association of genetic predisposition to CHIP with cirrhosis risk. To increase statistical power to detect an effect, we used variants associated with CHIP status at a p-value of less than 0.0001. We tested the association of these variants with cirrhosis risk using summary statistics from a genome wide association study of 5777 cirrhosis cases and 487780 controls. Emdin et al., A missense variant in Mitochondrial Amidoxime Reducing Component 1 gene and protection against liver disease, bioRxiv, doi:doi.org/10.1101/594523 (2019). We used the Mendelian randomization with robust adjusted profile score method, which provides for control of type 1 error rate when using sub-genome wide significant genetic variants. All statistical analyses were conducted using R version 3.5. Zhao et al., Statistical inference in two-sample summary-data Mendelian randomization using robust adjusted profile score, arXiv:1801.09652 (2018).

For murine studies, pairwise comparisons were made using Student's t-test for parametric data and Mann Whitney U test for non-parametric data. Statistical analyses were performed in Graphpad Prism 8.

B. Results

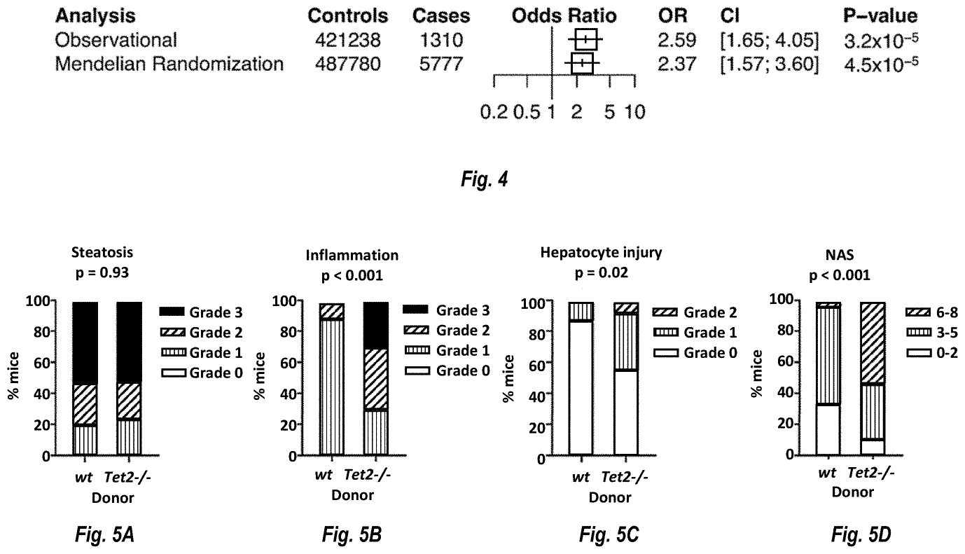

To assess the causality of the association of CHIP status with chronic liver disease, we performed Mendelian randomization analysis. We identified 184 independent variants associated with CHIP status from a recent GWAS comprised of 97,691 blood DNA-derived whole genome sequences at p<0.0001 significance. Bick et al., Inherited causes of clonal hematopoiesis of indeterminate potential in TOPMed whole genomes, bioRxiv doi:doi.org/10.1101/782748 (2019). We tested the association of these variants with cirrhosis risk using summary statistics from a genome wide association study of 5777 cirrhosis cases and 487780 controls. Emdin et al., A missense variant in Mitochondrial Amidoxime Reducing Component 1 gene and protection against liver disease, bioRxiv, doi:doi.org/10.1101/594523 (2019). Using the Mendelian randomization with robust associated profile (MR-RAPS) method, which increases power to detect a significant effect of genetic predisposition to CHIP and accounts for potential directional pleiotropy, CHIP was associated with a three-fold risk of chronic liver disease (OR 3.20 CI 1.95, 5.24, p<0.001). This estimate did not differ significantly from the observational estimate (p interaction=0.78, FIG. 4). Similar results were obtained in sensitivity analyses using the pleiotropy-robust Mendelian randomization methods median regression and MR-PRESSO, as well as using MR-RAPS with varying p-value thresholds for SNP instrument inclusion.

### Example 3b: Association of a Missense IL6R Variant with Chronic Liver Disease by CHIP Status

A. Results

Prior analyses suggest that a damaging interleukin 6 receptor gene (IL6R) missense variant (p.Asp358Ala, rs2228145) associates with greater protection from coronary artery disease among individuals with CHIP carriers versus non-CHIP carriers. Bick et al., Genetic interleukin 6 signaling deficiency attenuates cardiovascular risk in clonal hematopoiesis, Circulation 141:124-131 (2020). We therefore examined whether this variant is also associated with protection from chronic liver disease among individuals with CHIP.

We observed a significant interaction in the association of Asp358Ala with chronic liver disease by CHIP status (p_{interaction}=0.03). Asp358Ala was not associated with chronic liver disease risk among those without CHIP (OR 1.17 CI 0.91, 1.50, p=0.21). In contrast, Asp358Ala protected against chronic liver disease among those with CHIP (OR 0.32 CI 0.11, 0.95, p=0.04).

### Example 4: Tet2^{-/-} Hematopoiesis and Western Diet Induce Steatohepatitis in Mice

A. Methods

1. Mouse Models

Lethally irradiated Ldlr^{-/-}, Nlrp3^{-/-} and B6.SJL mice were transplanted with bone marrow cells from vavCre⁺Tet2^{fl/-} and control vavCre⁺ mice. Transplanted mice were fed an atherogenic Western diet containing 0.2% cholesterol and 42 kcal % fat (TD.88137; Envigo) or a choline-deficient, L-amino acid defined, high-fat diet containing 60 kcal % fat and 0.1% methionine CDAHFD, Research Diets). Perfused mouse livers were harvested and processed for histologic grading of steatohepatitis, fibrosis quantification by image analysis, RNA extraction and transcriptome analysis, and fluorescence-activated cell sorting of liver macrophages.

Ldlr^{-/-} and B6.SJL mice at 8 weeks were exposed to 10 Gy total body irradiation and transplanted with 1,000,000 to 2,000,000 bone marrow cells harvested from sex-matched vavCre⁺Tet2^{fl/-}, vavCre⁺Tet2^{fl/-}Nrp3^{fl/fl}, and control vavCre⁺ mice. After hematopoietic reconstitution was confirmed by peripheral blood analysis and flow cytometry at 4 weeks, mice were fed an atherogenic Western diet containing 0.2% cholesterol and 42 kcal % fat (TD.88137; Envigo) or a choline-deficient, L-amino acid defined, high-fat diet containing 60 kcal % fat and 0.1% methionine (CDAHFD, Research Diets) for defined time periods. All animal experiments were approved by the Institutional Animal Care and Use Committee (IACUC) at Brigham and Women's Hospital and Dana Farber Cancer Institute.

2. Mouse Peripheral Blood Analysis

Peripheral blood was collected from the retro-orbital sinus into EDTA tubes. Complete blood counts were obtained using a Hemavet 950 analyzer. After red cell lysis, cells were resuspended in PBS supplemented with 2% FBS for flow cytometric analysis using a BD FACScanto II analyzer. The following antibodies (all from Biolegend) were used for multicolor flow cytometry: mouse anti-mouse CD45.1 FITC (A20), mouse anti-mouse CD45.2 PE (104), rat anti-mouse CD3 PE-Cy7 (17A2), rat anti-human/mouse CD11b APC-Cy7 (M1/70), rat anti-mouse Gr-1 Pacific Blue (RB6-8C5), rat anti-human/mouse CD45R/B220 BV510 (RA3-6B2). Plasma was obtained by centrifugation at 1,000×g for 10 minutes at 4° C. and cytokine levels measured using Luminex-based mouse cytokine/chemokine magnetic bead panel (Millipore).

3. Histologic Assessment

Mouse livers were fixed in 10% formalin for 24 hours. Paraffin embedded tissue blocks were sectioned and stained using hematoxylin & eosin and Masson's trichrome for blinded grading of steatohepatitis and fibrosis, respectively, according to modified CRN criteria as shown below in Table 5. Liver fibrosis was measured by histologic grading of Masson's Trichrome or quantification of Picosirius Red positivity using ImageJ.

TABLE 5
Histologic grading criteria for steatohepatitis in mice
Steatosis

| | |
|---|---|
| Grade 0 | Absent |
| Grade 1 | <33% of hepatocytes |
| Grade 2 | 33-66% of hepatocytes |
| Grade 3 | <66% of hepatocytes |

Inflammation

| | |
|---|---|
| Grade 0 | Absent |
| Grade 1 | <100 inflammatory cells per focus |
| | or <3 inflammatory foci per 20x field |
| Grade 2 | 100-500 inflammatory cells per focus |
| | or 3-4 inflammatory foci per 20x field |
| Grade 3 | >500 inflammatory cells per focus |
| | or >5 inflammatory foci per 20x field |

Hepatocyte Injury/Ballooning

| | |
|---|---|
| Grade 0 | Absent |
| Grade 1 | Rare balloon cells |
| Grade 2 | Widespread hepatocyte ballooning and apoptosis |

Modified NASH activity score (NAS)

| | |
|---|---|
| Grade 1 | 0-2 points |
| Grade 2 | 3-4 points |
| Grade 3 | >5 points |

Modified from NASH Clinical Research Network scoring system for NAFLD (Kleiner et al., Design and validation of a histological scoring system for nonalcoholic fatty liver disease, Hepatology 41(6): 1313-21, doi: 10.1002/hep.70701 (2005)).

B. Results

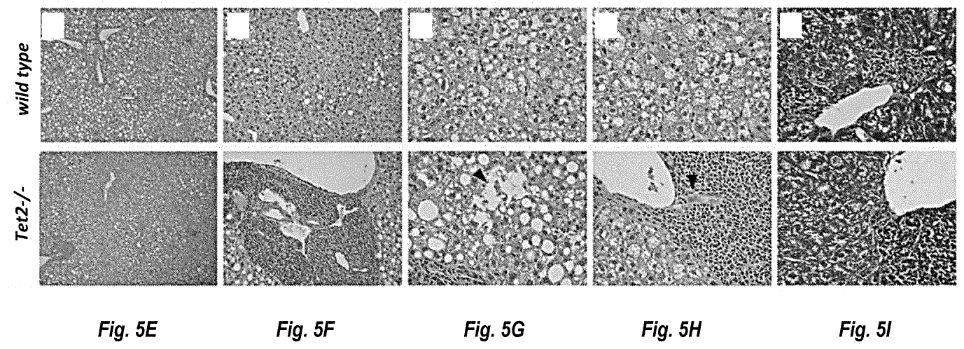

To examine whether mutant hematopoietic cells can influence liver injury, Ldlr^{-/-} mice transplanted with Tet2^{-/-} or control bone marrow cells were fed Western diet for 9 weeks. To examine liver fat and inflammation, we performed qualitative histologic scoring using a modified NAFLD activity score (NAS) (FIGS. 5A-D). (Kleiner et al., Design and validation of a histological scoring system for nonalcoholic fatty liver disease, Hepatology 41(6):1313-21, doi:10.1002/hep.70701 (2005). Mice with wild-type or Tet2^{-/-} hematopoietic cells had similar hematologic parameters and liver fat accumulation (FIG. 5A-I). In contrast, portal and lobular inflammation as well as hepatocyte ballooning injury were significantly increased in Tet2^{-/-} transplanted animals (FIGS. 5F-H). Development of fibrosis was not observed in Tet2^{-/-} and control animals during the short duration of diet in this experiment (FIG. 5I). These findings demonstrate that Tet2^{-/-} hematopoietic cells transplanted into Ldlr^{-/-} mice and fed Western diet increase steatohepatitis by clinically relevant criteria.

### Example 5: Effect of Tet2^{-/-} Hematopoiesis on Diet-Induced Non-Alcoholic Steatohepatitis (NASH)

A. Methods

1. Mouse Models, Mouse Peripheral Blood Analysis, and Histologic Assessment

Mouse models, mouse peripheral blood analysis, and gistologic assessment methods were carried out as described in Example 4 above.

B. Results

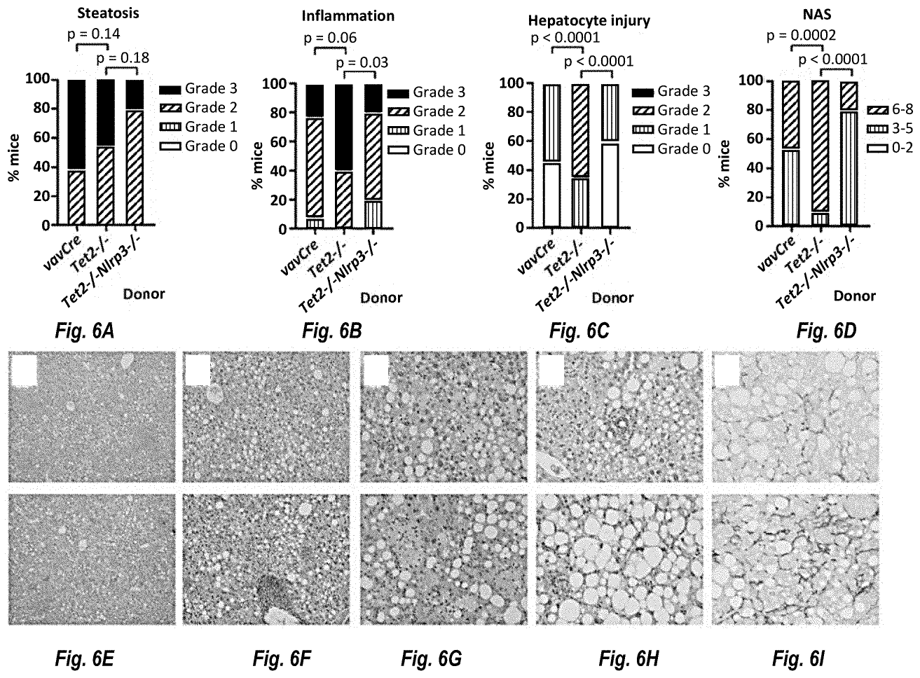

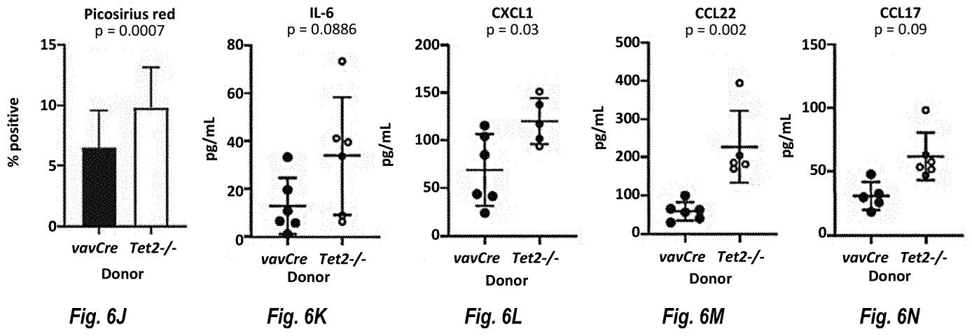

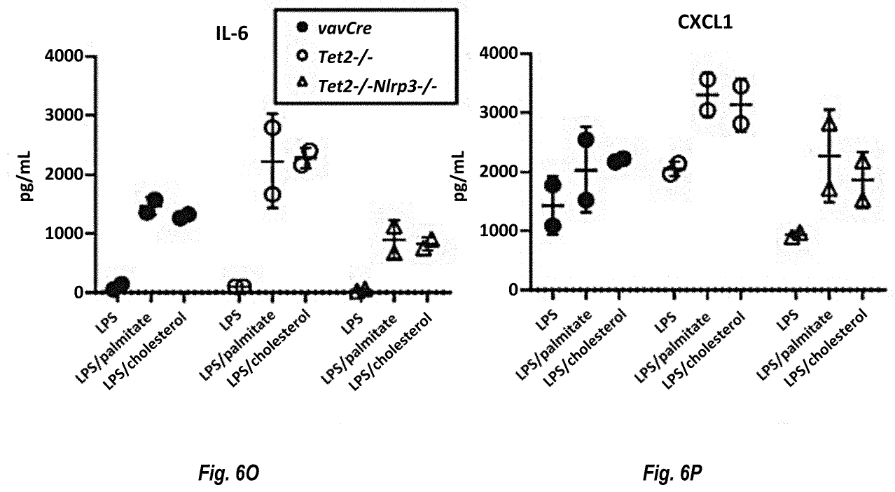

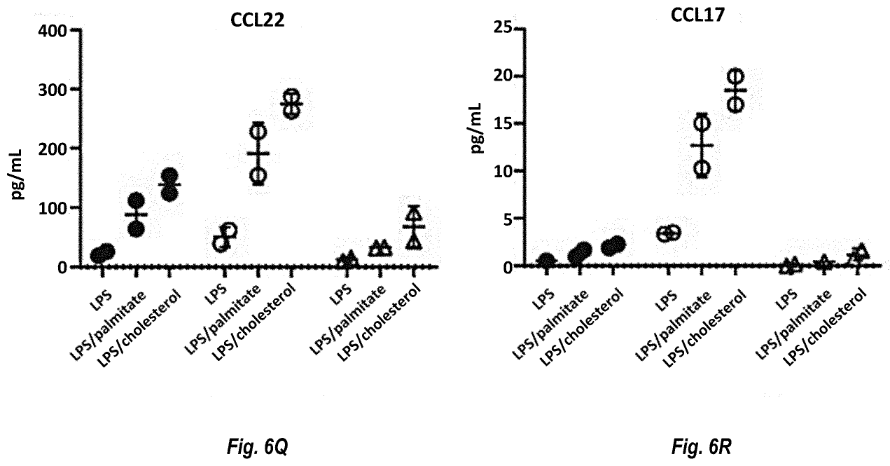

We next explored the impact of hematopoietic-specific Tet2 inactivation in a dietary model of non-alcoholic steatohepatitis that recapitulates the cardinal features of human disease. Ikawa-Yoshida et al., Hepatocellular carcinoma in a mouse model fed a choline-deficient, L-amino acid-defined, high-fat diet, Int. J. Exp. Pathol. 98(4):221-233, doi:10.1111/iep.12240 (2017); Matsumoto et al., An improved mouse model that rapidly develops fibrosis in non-alcoholic steatohepatitis, Int. J. Exp. Pathol. 94(2):93-103, doi:10.0000/iep.12008 (2013). As in the Ldlr^{-/-} experiments, mice transplanted with Tet2^{-/-} or Dnmt3a^{-/-} bone marrow and fed a choline deficient, high fat diet (CDAHFD) developed increased liver inflammation and hepatocyte injury, resulting in higher overall NASH activity (FIGS. 6A-H and 8A-E). In comparison, liver inflammation and hepatocyte injury were significantly reduced in mice transplanted with Tet2^{-/-}Nlrp3^{-/-} bone marrow cells (FIGS. 6A-H). Therefore, hematopoietic loss of Tet2 promotes diet induced steatohepatitis via NLRP3 inflammasome.

Because significant liver fibrosis was not observed at the initial endpoint, mice were fed CDAHFD for an extended duration. At 19 weeks, Tet2^{-/-} transplanted mice showed increased liver fibrosis compared to wild type controls (FIGS. 6I and 6J), confirming that hematopoietic loss of Tet2 promotes liver fibrosis in CDAHFD fed mice.

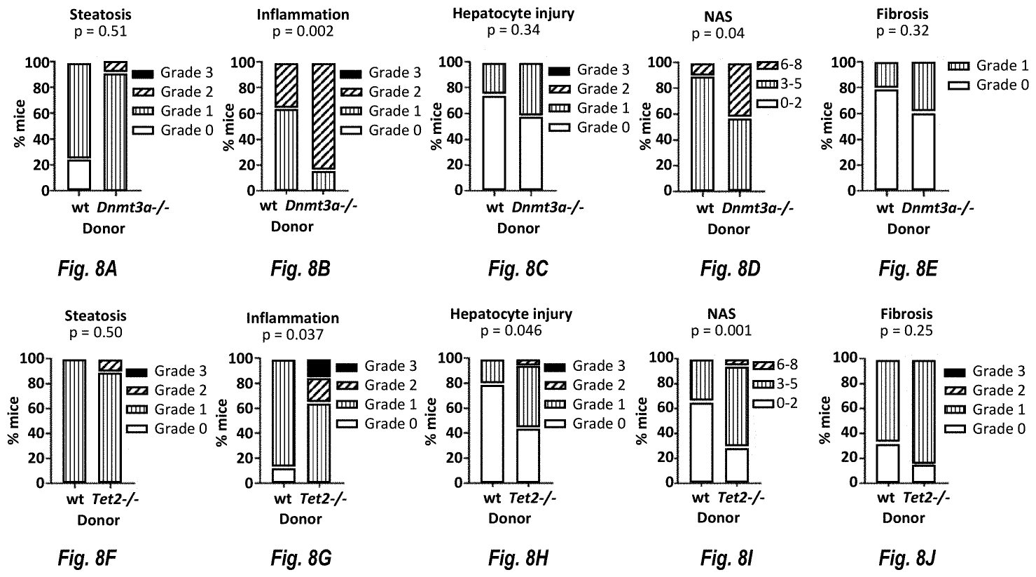

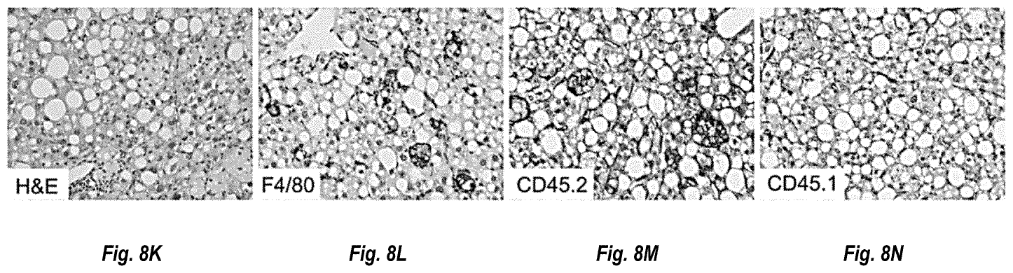

To study the persistence of liver injury in the setting of mutant hematopoiesis, Tet2^{-/-} transplanted animals were fed CDAHFD for 11 weeks, then subsequently fed regular chow for 10 days. Although we observed a global decrease in steatohepatitis after diet reversion as levels in Tet2^{-/-} transplanted mice (FIGS. 8F-J). In contrast, liver fat regressed at a similar rate in control. These findings indicate that the prolongation of liver inflammation and injury by Tet2^{-/-} hematopoietic cells may promote the development of NASH in the setting of repeated liver insults. Buzzetti et al., The multiple-hit pathogenesis of non-alcoholic fatty liver disease (NAFLD), Metabolism 65(8):1038-48, doi:10.1016/j.metabol.2015.12.012 (2016).

### Example 6: Pro-Inflammatory Role of Tet2 in Monocyte-Derived Liver Macrophages

a. Methods

1. Bone Marrow Derived Macrophages

Bone marrow cells from vavCre⁺Tet2^{fl/-}, vavCre⁺Tet2^{fl/-}Nlrp3^{fl/fl}, and control vavCre⁺ mice cultured with recombinant M-CSF (Preprotech) for 8 days and terminal differentiation confirmed using flow cytometry. Macrophages were exposed to 10 ng/mL lipopolysaccharide (Sigma) for 2 hours and stimulated with palmitic acid (300 μM) or cholesterol monohydrate crystals (200 μg/mL) for 6 hours. Culture supernatant was centrifuged at 1,000×g for 10 minutes at 4° C. and cytokine levels were measured using Luminex-based cytokine-chemokine magnetic bead panel (Millipore).

2. Mouse Models and Liver Histology

Mouse models and liver histologic assessment used in this Example are as described above in Example 4.

3. Cell Sorting

Freshly perfused liver tissue was diced and digested with 850 mg/mL collagenase I, 700 mg/mL collagenase D, 1 mg/mL Dispase II and 100 ng/mL DNase I for 40 minutes in an orbital shaker at 37° C. and filtered through a 70-micron mesh filter. After red cell lysis, cells were resuspended in PBS supplemented with 2% FBS for flow cytometric analysis using a Sony MA900 cell sorter. The following antibodies (all from Biolegend) were used for multicolor flow cytometry: rat anti-mouse F4/80 FITC (BM8), rat anti-mouse Ly6C APC (HK1.4), rat anti-mouse CD3 PerCP-Cy5.5 (17A2), rat anti-human/mouse CD45R/B220 PerCP-Cy5.5 (RA3-6B2), mouse anti-mouse NK1.1 PerCP-Cy5.5 (PK136), rat anti-human/mouse CD11b APC-Cy7 (M1/70), rat anti-mouse Ly6G PE-Cy7 (1A8), mouse anti-mouse CD45.2 Pacific Blue (104), mouse anti-mouse CD45.1 BV510 (A20).

4. RNA Analysis

RNA from freshly perfused mouse liver or sorted liver macrophages were extracted using Qiagen RNeasy Plus kit. Fragmentation, reverse transcription and cDNA library preparation with random hexamer primers were performed using standard Illumina protocols. Pooled libraries were sequenced using Illumina NovaSeq 6000. Sequenced reads were filtered to remove reads containing adapters, greater than 10% undetermined bases, or greater than 50% of bases with Q score less than or equal to 5. Transcript abundance estimates were generated using Salmon v1.2.1 and differentially expressed genes (log 2FC>0.58, padj<0.05) identified using R package DESeq2. Gene set enrichment analysis was carried out using GSEA v4.1.0 (Broad Institute). For sorted liver macrophages, gene sets in MSigDB C7 (filtered for macrophage-related signatures) and H (Hallmark) collections were analyzed. For unsorted liver transcripts, gene sets in MSigDB C2 (filtered for liver-related signatures) and H collections were analyzed, in addition to liver disease-specific gene sets extracted from published data. Hoang et al., Gene Expression Predicts Histological Severity and Reveals Distinct Molecular Profiles of Nonalcoholic Fatty Liver Disease. Sci Rep 2019; 9(1):12541l; Gerhard et al., Transcriptomic Profiling of Obesity-Related Nonalcoholic Steatohepatitis Reveals a Core Set of Fibrosis-Specific Genes. J Endocr Soc 2018; 2(7):710-26; Ryaboshapkina et al., Human hepatic gene expression signature of non-alcoholic fatty liver disease progression, a meta-analysis. Sci Rep 2017; 7. P value<0.05 and FDR<0.15 were taken to be significant.

b. Results

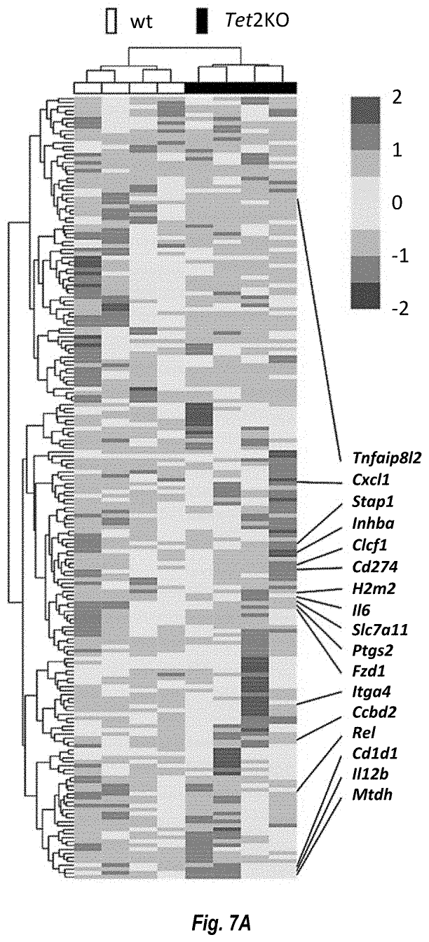

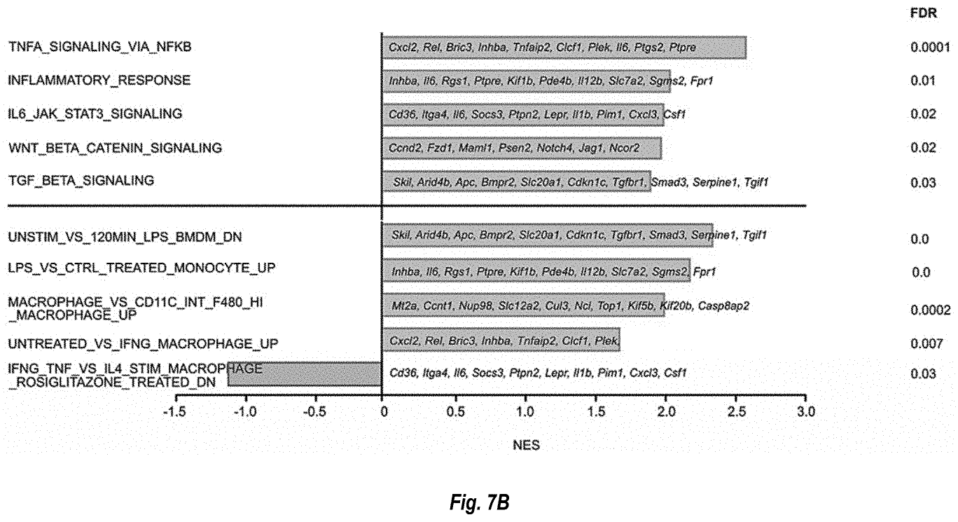

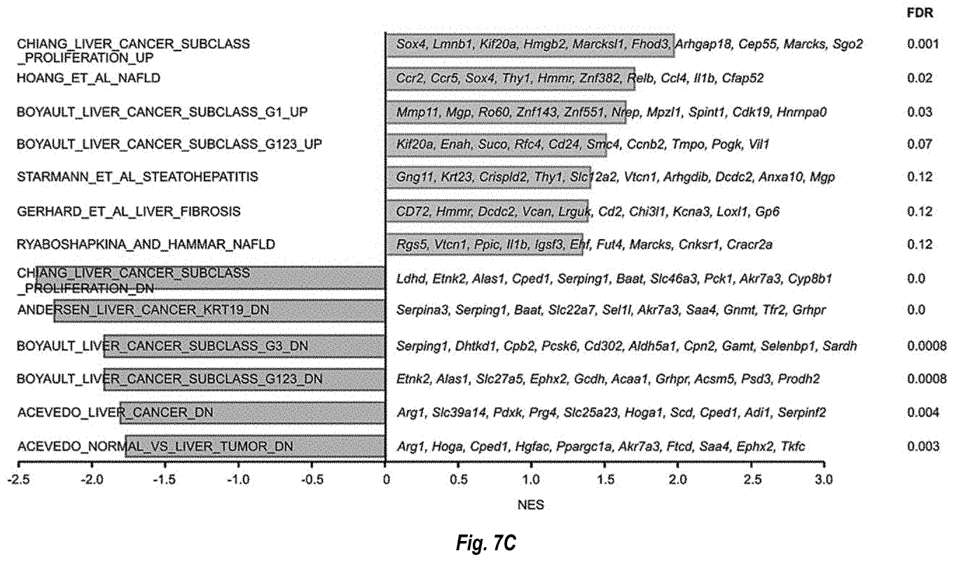

Kupffer cells constitute the majority of liver resident macrophages and are replaced by bone marrow derived macrophages after hematopoietic transplant (FIGS. 8K-N). Klein et al., Kupffer cell heterogeneity: functional properties of bone marrow derived and sessile hepatic macrophages, Blood 110(12):4077-85 (2007), doi10.1182/blood-2007-02-073841. Unlike Kupffer cells, which express CD45⁺F4/80⁺CD11b^{int}, hematopoietic derived liver macrophages can be characterized as CD45⁺F4/80^{mod}CD11b^{hi}. Scott et al., Bone marrow-derived monocytes give rise to self-renewing and fully differentiated Kupffer cells, Nat. Commun. 7, 10321 (2016), doi:10.1038/ncomms10321. We performed RNA sequencing on CD45⁺F4/80^{mod}CD11b^{hi} liver macrophages in mice transplanted with Tet2^{-/-} or wild type marrow and fed CDAHFD. Compared to wild type cells, Tet2^{-/-} macrophages showed increased expression of Il-6 and Cxcl1 among other pro-inflammatory genes (FIG. 7A). Consistent with gene expression analysis, mice transplanted with Tet2^{-/-} hematopoietic cells showed increased serum levels if IL-6, CXCL1, CCL22, and CCL17 (FIGS. 6K-R). CXCL1 and IL-6 are pro-inflammatory molecules regulated by the NLRP3 inflammasome complex, whereas chemokines CCL17 and CCL22 promote the recruitment of regulatory T cells. Oo et al., J Immunol 184:2886-2898 (2010). Confirming the notion that proinflammatory effect of hematopoietic Tet2 loss is mediated via NLRP3 dependent responses, macrophages derived from bone marrow cells lacking both Tet2 and Nlrp3 showed baseline expression levels of IL-6, CXCL1, CCL22, and CCL17 compared to Tet2^{-/-} cells (FIGS. 6O-R).

Gene set enrichment analysis further revealed that Tet2^{-/-} liver macrophages exhibit inflammatory responses that are mediated primarily by IL-6 and TNFα signaling, as well as enhanced profibrotic pathways via TGFβ and WNT signaling (FIG. 7B). In addition, liver mRNA from mice transplanted with Tet2^{-/-} marrow show enrichment in transcriptional programs associated with steatohepatitis and hepatocellular carcinoma (FIG. 7C). Together, these findings support a model in which Tet2 deficient liver macrophages induce pro-inflammatory signals that promote steatohepatitis.

### Discussion

We have demonstrated that CHIP is associated with an elevated risk of both prevalent and incident chronic liver disease, including alcohol-related liver disease and obesity-associated liver disease. Among middle-aged adults without liver disease in the UK Biobank, 1 in 20 individuals with CHIP developed chronic liver disease by 80 years of age, compared to only 1 in 100 individuals without CHIP. The overall nearly four-fold risk of incident chronic liver disease observed in the current study is greater than the nearly two-fold risk of incident coronary artery disease previously reported with CHIP in overlapping cohorts. Jaiswal et al., Clonal hematopoiesis and risk of atherosclerotic cardiovascular disease, N. Engl. J. Med. 377:111-121 (2017); Bick et al., Genetic interleukin 6 signaling deficiency attenuates cardiovascular risk in clonal hematopoiesis, Circulation 141:124-131 (2020). Mendelian randomization and murine models support a causal role for CHIP in chronic liver disease pathogenesis. Furthermore, stratified Mendelian randomization analyses and ex vivo liver macrophage inflammatory biomarker analyses implicate IL-6 and more broadly, NLRP3 inflammasome in CHIP-associated chronic liver disease.

Our findings support a model of CHIP promoting steatohepatitis particular among individuals with elevated liver fat or other sources of liver injury that increase the risk of cirrhosis.

First, individuals with CHIP showed higher indices of liver inflammation and fibrosis with no difference in liver fat accumulation. Second, hematopoietic-specific Tet2 inactivation increased the severity of diet-induced steatohepatitis in mice due to increased liver inflammation and hepatocyte injury, without influence on liver fat. In contrast to germline genetic variants which predispose to both liver fat and cirrhosis (Emdin et al., A missense variant in Mitochondrial Amidoxime Reducing Component 1 gene and protection against liver disease, bioRxiv, doi:doi.org/10.1101/594523 (2019)), these findings suggest that clonal hematopoiesis confers a pro-inflammatory drive to activate local immune and fibrogenic pathways in the fatty liver. Liver fat accumulation may elicit different inflammatory responses in normal versus mutant hematopoietic cells.

We also provide human genetic evidence for a causal relationship between CHIP and liver disease. Mendelian randomization analysis may be useful to distinguish outcomes that are causally related to CHIP from confounding associations. To optimize statistical power in the setting of low heritability of CHIP (Bick et al., Inherited causes of clonal hematopoiesis of indeterminate potential in TOPMed whole genomes, bioRxiv doi:doi.org/10.1101/782748 (2019)), we applied a newly developed Mendelian randomization technique (MR-RAPS) which allows for use of sub-genome wide significant variants. Zhao et al., Statistical inference in two-sample summary-data Mendelian randomization using robust adjusted profile score, arXiv:1801.09652 (2018). We observed that germline genetic predisposition to CHIP also predisposes to chronic liver disease risk. Therefore, CHIP is likely a causal risk factor for liver disease. Consequently, targeting factors that promote CHIP-associated liver injury as well as the prevention of CHIP itself are expected to reduce the risk of chronic liver disease among susceptible individuals.

We previously reported that genetic deficiency of IL-6 signaling due to the presence of IL6R p.Asp358Ala was associated with a markedly reduced risk for incident cardiovascular disease risk, but not hematologic malignancy specifically, among individuals with CHIP. Bick et al., Genetic interleukin 6 signaling deficiency attenuates cardiovascular risk in clonal hematopoiesis, Circulation 141:124-131 (2020). In the current study, we also observed that the presence of IL6R p.Asp358Ala was associated with a greater reduction in chronic liver disease risk among individuals with CHIP compared to those without CHIP. Tet2^{-/-} liver macrophages in transplanted mice exhibited a significant increase in IL6 expression, as well as an enrichment in pro-inflammatory macrophage gene signatures. The expression of these proinflammatory cytokines and the pathogenesis of steatohepatitis were abrogated by the loss of NLRP3 inflammasome in Tet2 deficient hematopoietic cells specifically. Although the role of IL-6 in NAFLD and other chronic liver diseases remains controversial (Wunderlich et al., Cell Metab 12:237-249 (2010); Yamaguchi et al., Lab Invest 90:1169-1178 (2010)), broad pharmacologic inhibition of NLRP3 inflammasome reduces liver inflammation and fibrosis in mice. Mridha A R et al., J Hepatol 2017 66:1037-1046. These findings suggest that therapies targeting inflammatory pathways may be useful for prevention of chronic liver disease in individuals with CHIP.

In conclusion, CHIP is associated with an elevated risk of chronic liver disease specifically through the promotion of liver inflammation and injury. As with atherosclerotic cardiovascular disease, CHIP may also be a modifiable risk factor for chronic liver disease.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of treating liver disease in a human subject comprising treating the liver disease with lifestyle modifications and/or by administering an effective amount of at least one pharmaceutical agent for treating liver disease, wherein the human subject has a DNMT3A, TET2, JAK2, and/or ASXL1 mutation, thereby treating the liver disease.

2. A method of treating liver disease in a human subject comprising:
a. sequencing at least part of a genome comprising DNMT3A, TET2, JAK2, and/or ASXL1 of one or more cells in a blood sample of the human subject;
b. determining from the sequencing whether the human subject has one or more mutations in DNMT3A, TET2, JAK2, and/or ASXL1 and
c. if it is determined that the human subject has at least one DNMT3A, TET2, JAK2, and/or ASXL1 mutation, treating the liver disease with lifestyle modifications and/or by administering an effective amount of at least one pharmaceutical agent for treating liver disease, to the human subject thereby treating the liver disease.

3. The method of claim 1, wherein (a) the pharmaceutical agent for treating liver disease targets NLRP3 inflammasome, IL-1β, IL-6, IL-6 receptor, CCL22, Cxcl1, Ccl17, MCP1, and/or MIP2, and/or (b) the pharmaceutical agent is an inhibitor of NLRP3 inflammasome, IL-1β, IL-6, IL-6 receptor, CCL22, Cxcl1, Ccl17, MCP1, and/or MIP2.

4. (canceled)

5. The method of claim 1, wherein the pharmaceutical agent comprises:
a. an antibody or an antigen binding fragment thereof;
b. melatonin, methylprednisolone, AC-201, clazakizumab, tocilizumab, anakinra (Kineret®), canakinumab (Ilaris®), ziltivekimab, sarilumab, sinomenine, fucoidan, or bindarit;
c. a hepatitis treatment; and/or
d. a treatment to prevent ascites, edema, portal hypertension, severe bleeding, or infections.

6. (canceled)

7. (canceled)

8. (canceled)

9. (canceled)

10. The method of claim 1, wherein the method of treating liver disease comprises at least one lifestyle modification.

11. The method of claim 10, wherein the at least one lifestyle modification is chosen from weight loss, exercise, and dietary modification.

12. The method of claim 11, wherein dietary modification is chosen from cessation or reduction of alcohol consumption, a low sodium diet, a low fat diet, a low carbohydrate diet, a high fiber diet, and cessation or reduction of medications and/or supplements that are toxic to the liver.

13. A method for diagnosing liver disease, diagnosing CHIP, predicting risk for CHIP, and/or targeted prevention of liver disease in a human subject comprising:
a. obtaining a nucleic acid sample from the human subject;
b. detecting whether the sample contains at least one DNMT3A, TET2, JAK2, and/or ASXL1 mutation; and
c. diagnosing the human subject as having liver disease, CHIP, a risk for CHIP, and/or a risk of liver disease when at least one DNMT3A, TET2, JAK2, and/or ASXL1 mutation is detected.

14. The method of claim 13, wherein the at least one DNMT3A, TET2, JAK2, and/or ASXL1 mutation is a loss-of-function mutation.

15. The method of claim 13, wherein the at least one DNMT3A, TET2, JAK2, and/or ASXL1 mutation comprises an amino acid change in TET2 chosen from S145N, S282F, A308T, N312S, L346P, P399L, S460F, D666G, S817T, P941S, C1135Y, R1167T, I1175V, S1204C, R1214W, D1242R, D1242V, Y1245S, R1261C, R1261H, R1261L, F1287L, W1291R, K1299E, K1299N, R1302G, E1318G, P1367S, C1396W, L1398R, V1417F, G1869W, L1872P, I1873T, C1875R, H1881Q, H1881R, R1896M, R1896S, S1898F, V1900A, G1913D, A1919V, R1926H, P1941S, P1962L, R1966H, R1974M, and R2000K.

16. The method of claim 13, wherein the at least one DNMT3A, TET2, JAK2, and/or ASXL1 mutation comprises an amino acid change in JAK2 of V617F.

17. The method of claim 13, wherein a TP53, SF3B1, SRSF2, GNB1, CBL and/or PPM1D mutation is also detected in the human subject.

18. The method of claim 13, wherein at least one mutation is detected by a SNP array.

19. The method of claim 13, wherein the human subject does not have a mutation in NLRP3, IL-1β, and/or IL-6 that inhibits the NLRP3/IL-1β/IL-6 signaling blockade.

20. The method of claim 19, wherein the human subject does not have a D358A mutation in IL6R.

21. The method of claim 13, wherein the human subject also exhibits one or more risk factors comprising increased levels of cholesterol, increased levels of liver iron, increased alcohol consumption, increased body mass index, and/or obesity.

22. The method of claim 13, wherein the human subject does not have myeloproliferative neoplasm.

23. The method of claim 13, wherein the human subject does not have portal vein thrombosis.
